(19) United States
(12) Patent Application Publication
(10) Pub. No.: US 2004/0115720 A1
(43) Pub. Date: Jun. 17, 2004

(54) High throughput automatic nucleic acid isolation and quantitation methods

(76) Inventors: Diana R. McWilliams, Bonne Terre, MO (US); Robert G. Becker, Wildwood, MO (US); Charles W. Bolten, Kirkwood, MO (US); Pamela A. De Ciechi, O'Fallon, MO (US); Shawn D. Miller, Fenton, MO (US); Andrew T. Pinz, O'Fallon, MO (US); Jeffrey V. Surry, Florissant, MO (US)

Correspondence Address: Gardere Wynne Sewell LLP, Suite 3400, 1000 Louisiana, Houston, TX, 77002-5007, US

(21) Appl. No.: 10/703,908

(22) Filed: Nov. 7, 2003

Related U.S. Application Data
(60) Provisional application No. 60/425,136, filed on Nov. 8, 2002.
(60) Provisional application No. 60/425,139, filed on Nov. 8, 2002.

(51) Int. Cl. C12Q 1/68; G06F 19/00; G01N 33/48; G01N 33/50

(52) U.S. Cl. 435/6; 702/20

(57) ABSTRACT

A high throughput RNA laboratory protocol is provided for the extraction and maintenance of a sufficient quantity of high quality RNA during sample preparation in order to analyze several genes at a time with assistance of computer analysis. The subject invention includes a method for analyzing RNA comprising the steps of extracting RNA from a complex biological construct in sufficient quantities to provide accurate RNA data, transferring RNA to an apparatus that maintains the RNA and necessary reagents at a temperature of between about 0 to 10° C., and analyzing RNA with a computer generated mathematical analysis of the data to access the presence of RNA and ultimately test the efficacy of a drug.

### CROSS-REFERENCE TO RELATED APPLICATIONS

The present application claims priority under Title 35, United States Code, §119(e)(1) of U.S. Prov. Pat. Apps. Ser. Nos. 60/425,136 and 60/425,139, filed Nov. 8, 2002.

### BACKGROUND

A key area of pharmaceutical research is the determination of genetic expression. In vivo experimentation of pharmacological products mandates an accurate analysis of the cellular function and gene expression to determine efficacy and safety. The expression of a particular gene often indicates the efficacy or risk of administering the product to a patient.

The polymerase chain reaction (“PCR”) has revolutionized genetic research by providing a rapid means of amplifying and subsequently identifying specific nucleic acid sequences from complex genetic samples without the need for time-consuming cloning, screening and nucleic acid purification protocols. PCR was originally disclosed and claimed by Mullis et al. in U.S. Pat. Nos. 4,683,195, 4,683,202, and 4,965,188, hereby incorporated by reference. Since that time, considerable advances have been made in the reagents, equipment and techniques available for PCR. These advances have increased both the efficiency and utility of the PCR reaction, leading to its adoption to an increasing number of different scientific applications and situations.

The earliest PCR techniques were directed toward qualitative and preparative methods rather than quantitative methods. PCR was used to determine if a given DNA sequence was present in any quantity at all or to obtain sufficient quantities of a specific nucleic acid sequence for further manipulation. Originally, PCR was not typically employed to measure the amount of a specific DNA or RNA present in a sample. Only in recent years has quantitative PCR come to the forefront of nucleic acid research.

While DNA is necessary for PCR analysis, in testing the efficacy and safety of drugs, it is the mRNA that is the most accurate indicator of gene expression. There are many steps in the pathway leading from DNA to protein and all of them can in principle be regulated. A cell controls the proteins its makes by: 1) controlling when and how often a given gene is transcribed (transcriptional control), 2) controlling how the primary RNA transcript is spliced or other processed (RNA processing control), 3) selecting which completed mRNAs in the cell nucleus are exported to the cytoplasm (RNA transport control), 4) selecting which mRNAs in the cytoplasm are translated by ribosomes (translational control), 5) selectively destabilizing certain mRNA molecules in the cytoplasm (mRNA degradation control), or 6) selectively activating, inactivating or compartmentalizing specific protein molecules after they have been made (protein activity control). Molecular Biology of the Cell, 3^{rd} Ed. at 403. Although all of these steps involved in expressing a gene can in principle be regulated, for most genes, transcriptional controls are paramount and the initiation of RNA transcription is the most important point of control. Id. Therefore, mRNA is purified and cDNA clones produced to measure gene expression in the experimentation of pharmacological products.

Amplification of RNA into cDNA clones is accomplished by including a reverse transcription step prior to the start of PCR amplification. Reverse transcriptase (“RT”) is a DNA polymerase used to synthesize a cDNA strand using an mRNA template and primer, and is often used in conjunction with PCR in order to measure gene expression. This process is known as RT-PCR. By purifying mRNA, producing cDNA and amplifying the cDNA, gene expression is measured.

In a one-step RT-PCR process, reverse transcriptase, taq polymerase, primers, dNTPs and mRNA are added to the same tube and reverse transcription and amplification occur without further removal or addition of reagents. In two-step RT-PCR, reverse transcriptase, mRNA, dNTPs, and primers are used to make cDNA. The cDNA may be transferred to a new tube and primers, dNTPs, probes and Taq polymerase are then added together to amplify the DNA. The two-step protocol is prone to contamination because of the need to expose the samples to air while adding reagents.

Moreover, the reverse transcriptase is a temperature sensitive enzyme that begins to degrade above approximately 10° C. While optimal activity of the enzyme occurs at 37 to 48° C., the enzyme quickly degrades at this temperature. Even though reverse transcription is performed between 37 to 48° C., the reverse transcriptase looses activity during prolonged periods of elevated temperature. Reverse transcriptase maintains activity for at least 8 hours when stored at 4° C. However, activity may be lost within 30 minutes at a temperature of 48° C.

Once at room temperature, mRNA may denature if not used immediately as RNA degrades when exposed to heat or high pH. RNA degradation by alkaline hydrolysis is accelerated by heat. While RNase inhibitors may be added to protect the mRNA, RNase contamination may occur and degrade the mRNA. If RNA is degraded, an inaccurate analysis may result. Hence, maintaining RNA at a low temperature minimizes degradation.

Also, at room temperature, taq polymerase may activity may begin prior to the start of PCR. When this occurs, the yield and specificity of PCR is decreased at least partially due to the priming (or mis-priming) of sequences. Hence, premature taq polymerase activity provides inaccurate results in the analysis of genetic expression.

In the busy high throughput RNA laboratory, reverse transcriptase, taq polymerase, primers, dNTPs, mRNA and other constituents are often added simultaneously to numerous racks of tubes and/or plates. Often times and for a number of reasons there is a delay in amplifying and subsequently identifying specific nucleic acid sequences from complex genetic samples via the RT PCR reaction. Having plates and racks of tubes standing waiting for amplification at room temperature is very likely to taint the results of the expression analysis.

Moreover, regardless of the method used, the end result is the same, a plot of fluorescence versus cycle number is required. Further analysis of this data is then used to derive quantitative values for the RNA's present in the samples. Successful amplification of the sample will result in a sigmoidal plot consisting of a period where amplification is not detectable above the background noise of the experiment, a period of exponential amplification and a period where amplification plateaus. To analyze the data, threshold value is selected that is greater than the background noise of the experiment. Each amplification curve is analyzed to determine the point at which the curve rises above the threshold values. This is recorded in terms of the cycle in which this occurred and is known as the threshold cycle (C_{T}).

As originally published in User Bulletin #2 for ABI Prism 7700 Sequence Detection System, incorporated herein by reference, in the linear range (or exponential phase) the threshold cycle is inversely proportional to the amount of RNA in a sample. These values can be compared to a plot of threshold cycles obtained from amplification of serial dilutions of an exogenously added standard to determine the concentration RNA in the experimental samples. If the absolute quantity of the exogenously added standard is known, the absolute quantities of RNA in the experimental samples can be determined. However, the standard can also be of unknown concentration, in which case, relative quantification will be obtained.

The use of standard curves requires the amplification of exogenously added nucleic acids, increasing the total number of amplifications required and lowering the throughput of the experiment. Furthermore, because of variations in the quantity and quality of nucleic acids between different samples, it is often beneficial to compare the amount of nucleic acid to an endogenous control. If an endogenous control is present, relative quantitation can be accomplished by mathematical analysis of the differences in cycle threshold between the experimental sample and the endogenous control, eliminating the need for standard curves and reducing the total number of amplification required in an experiment. This mathematical analysis is performed by the human investigator and can take weeks to prepare, publish and analyze.

Au automated way of preparing the data for analysis to meet the high-throughput requirements of today's drug discovery process is lacking. Moreover, an effective and efficient way of preparing and analyzing the results of a high throughput experiment to detect specific DNA or RNA transcripts is absent.

Hence, most laboratories focus on isolating a lot of RNA in order to satisfy the needs of the microarray group. However, for a busy laboratory, this is not practical. A need exists therefore for a high throughput isolation and analysis protocol used in the laboratory to produce a sufficient yield of high quality RNA in order to accurately analyze via automated means several genes at one time.

### SUMMARY OF THE INVENTION

A high throughput RNA laboratory protocol is provided for the extraction and maintenance of a sufficient quantity of high quality RNA during sample preparation and accurate results and analysis of several genes at one time. The subject invention is a method of analyzing RNA comprising the steps of extracting RNA from a complex biological construct in sufficient quantities to provide RNA data, transferring the RNA to an apparatus that maintains the RNA and necessary reagents at a temperature of between about 0 to 10° C., and analyzing the RNA levels and function with a computer generated mathematical analysis of the data. The complex biological construct may be either pulverized or liquefied. RNA is subsequently isolated and purified in an automated nucleic acid workstation.

The high throughput RNA laboratory of the subject invention comprises an apparatus for extracting and isolating nucleic acids from a complex biological construct, an apparatus for maintaining said RNA samples at a temperature of between about 0 to 10° C., and a computer readable program for use in connection with an information display apparatus wherein said computer readable program causes a computer to calculate and display cycle threshold valves, a delta C_{T}, a delta delta C_{T} and a relative transcription change (XRel) of said RNA sample. The laboratory of the subject invention may also preferably includes an automated nucleic acid workstation for isolating mRNA from said complex biological construct and an automatic liquid-handling apparatus for preparing RNA samples for reverse transcription and PCR amplification. The high throughput RNA laboratory may also include a real-time quantitative PCR amplification system.

The apparatus for the extraction and isolation of genetic molecules such as DNA, RNA, mRNA, rRNA or tRNA from an animal for use in the analysis of genetic expression comprises a component for rupturing the cells of the complex biological construct, a chamber for holding said complex biological construct wherein the chamber is designed to allow free movement of said component through chamber, and a means for applying force to the chamber wherein the complex biological construct is liquefied or pulverized releasing genetic molecules intact.

Apparatus for maintaining the RNA sample at a temperature of between about 0 to 10° C. include a novel metal block having a plurality of wells where each well has an open cylindrical upper end and a closed conical lower end and accommodates a biological sample receptacle having substantially the same shape as said well. Each well maintains the temperature of a biological sample in the receptacle during sample set-up and prior to reverse transcriptase and polymerase chain reaction analysis and is useful in connection with an automated liquid handling device.

Another apparatus that may be used for maintaining the RNA sample at a temperature of between about 0 to 10° C. comprises an incubator, a quantitative analysis machine, and a transfer mechanism for automated transfer of a plate to and from the incubator and to and from the quantitative analysis machine (“the mechanism”). The plate (sometimes referred to as a “microplate”) is maintained in a queue in the incubator prior to analysis in the quantitative machine at a temperature below about 10 degrees centigrade. The mechanism moves the plate from a liquid handling device, or from a plate stacker where the plate may be in queue, and transfers the plate to the incubator. Subsequently, the plate is removed from the incubator by the mechanism and placed into a quantitative analysis machine.

The laboratory of the subject invention also has computer software for analyzing an experiment to detect RNA from a two-dimensional plate configuration. A computer-readable medium contains instructions for controlling a computer system in the analysis of an experiment to detect RNA in a sample. The computer usable medium has a computer readable program code embodied therein for determining the presence of RNA in a sample contained within a dye layer of a well of a plate. A program storage device readable by a computer, tangibly embodies the program of instructions is executed by the computer and performs the method steps for analyzing the presence of RNA in a sample. Also provided is a computer-readable medium containing a data structure. A memory for storing data for access by the computer program comprises the data structure.

The combination of extracting RNA from a complex biological construct, maintaining the temperature of the RNA and reagents between about 0 to 10° C., and preparing the analysis with the assistance of computer software provides for an extremely efficient high throughput RNA laboratory. The focus of this laboratory is not on extracting high quantities of RNA for sampling. But rather, sufficient quantities of high quality RNA for multiple gene transcripts are needed and analyzed quickly.

### DETAIL DESCRIPTION OF THE DRAWINGS

For better understanding of the invention and to show by way of example how the invention may be carried into effect, reference is now made to the detail description of the invention along with the accompanying figures in which corresponding numerals in the different figures refer to corresponding parts and in which:

FIG. 1 is a logic flow diagram depicting the overall methodology used in a computer system of the present invention for analyzing an experiment to detect RNA or DNA from a two-dimensional plate configuration.

FIG. 2 is a logic flow diagram depicting step 1, experiment information, of the overall methodology used in the computer system.

FIG. 3 is a logic flow diagram depicting step 2, plate information, of the overall methodology used in the computer system.

FIG. 4 is a logic flow diagram depicting step 3, plate layout, of the overall methodology used in the computer system.

FIG. 5 is a logic flow diagram depicting step 4, group information, of the overall methodology used in the computer system.

FIG. 6 is a logic flow diagram depicting step 5, file information, of the overall methodology used in the computer system.

FIG. 7 is a logic flow diagram depicting step 6, raw data and outliner management, of the overall methodology used in the computer system.

FIG. 8 is a logic flow diagram depicting step 7, calculation, of the overall methodology used in the computer system.

FIG. 9 is a logic flow diagram depicting step 8, publish, of the overall methodology used in the computer system.

FIG. 10 depicts a cross-sectional view of a sealed chamber with grinding element.

FIG. 11 depicts a perspective view of a sealed chamber with liquefying/pulverizing component.

FIG. 12 depicts a perspective view of a freezer mill suitable for use in connection with the subject invention.

FIG. 13 depicts a perspective view of a mixer mill suitable for use in connection with the subject invention.

FIG. 14 depicts a perspective view of a tissue crusher suitable for use in connection with the subject invention.

FIG. 15 is an overall flow diagram of a first embodiment of the high throughput RNA laboratory of the subject invention.

FIG. 16 is flow diagram of the preparation of liquefied tissue.

FIG. 17 is a flow diagram of the ABI 6700 Nucleic Acid Preparation machine.

FIG. 18 is a flow diagram of the ABI 6100 Nucleic Acid Preparation machine.

FIG. 19 is a flow diagram of the process of preparing the sample for Taqman analysis.

FIG. 20 is a flow diagram of the RNA analysis prepared on the ABI 7900 or ABI 7700 machines.

FIG. 21A is a perspective view of the metal block suitable for polypropylene tubes.

FIG. 21B is a perspective view of the metal block suitable for a 96 well format.

FIG. 22 is an exploded view of the metal block and biological sample receptacles.

FIG. 23 is a cross-sectional view of the metal block.

FIG. 24 is a perspective view of a liquid handling device suitable for use in connection with the subject invention.

### DETAIL DESCRIPTION OF THE INVENTION

The subject invention is a high throughput RNA laboratory applying a novel method of analyzing RNA. In the laboratory of the subject of invention, the method comprises the steps of extracting RNA from a complex biological construct in sufficient quantities to provide accurate RNA data, transferring the RNA to an apparatus that maintains the RNA and necessary reagents at a temperature of between about 0 to 10° C., and analyzing the RNA levels and function with a computer generated mathematical analysis of the data. The complex biological construct may be either pulverized or liquefied. RNA is subsequently isolated and purified in an automated nucleic acid workstation.

The high throughput RNA laboratory of the subject invention comprises an apparatus for extracting and isolating nucleic acids from a complex biological construct, an apparatus for maintaining said RNA samples at a temperature of between about 0 to 10° C., and a computer readable program for use in connection with an information display apparatus. The computer readable program causes a computer to calculate and display cycle threshold valves, a delta C_{T}, a delta delta C_{T} and a relative transcription change (XRel) of the RNA sample. The laboratory of the subject invention may also include an automated nucleic acid workstations for isolating mRNA from the complex biological construct and an automatic liquid-handling apparatus for preparing RNA samples for reverse transcription and PCR amplification. The high throughput RNA laboratory further includes a real-time quantitative PCR amplification system.

As described in U.S. patent application Ser. No. 60/360,136 filed Feb. 26, 2002, U.S. patent application Ser. No. 60/411,174 filed Sep. 17, 2002, U.S. patent application Ser. No. 60/411,175 filed Sep. 17, 2002 and U.S. patent application Ser. No. unassigned filed October/November __, 2002, incorporated herein in their entirety, and to facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used throughout the present specification the following abbreviations are used:

C_{T} means threshold cycle value and is the cycle during PCR when there is a detectable increase in signal intensity or fluorescence above baseline.

CV means the coefficient of variation that is calculated for each set of replicate wells having the same group label, sample ID, and gene.

ΔC_{T} (also referred to as “delta C_{T}”)=Mean (C_{T} values for sample FPR)—Mean (C_{T} values Endogenous Control FPR).

ΔC_{T} Mean Vehicle (comparator group)=Mean (ΔC_{T} for all amplifications of the FPR set in the comparator group).

ΔC_{T} Median Vehicle (comparator group)=Median (ΔC_{T} for all amplifications of the FPR set in the comparator group).

ΔΔC_{T} (also referred to as “delta delta C_{T}”)=(ΔC_{T} for the sample, treated or diseased)—ΔC_{T} Median Vehicle (comparator group).

E means to the efficiency of amplification for each experiment and is assumed to be 1 (one).

FPR set means Forward Primer, Probe, and Reverse Primer Set used to identify the presence of a gene.

-RT means Minus Reverse Transcriptase, an amplification used to determine if DNA contaminants exist in the RNA. A -RT well contains RNA and an FPR set, but does not contain reverse transcriptase. Minus reverse transcriptase wells are related to sample wells that have the same RNA and FPR set as the -RT well.

NTC means no template control and is a well that contains no RNA.

PCR means polymerase chain reaction.

Rₙ, normalized reporter signal and is determined to be the signal activity of the reporter dye divided by the signal activity of the passive reference dye.

RT means reverse transcriptase.

XRel means relative transcriptional change or relative expression level of the gene.

Additional terms as used through the specification are defined as follows:

Amplify when used in reference to nucleic acids refers to the production of a large number of copies of a nucleic acid sequence by any method known in the art. Amplification is a special case of nucleic acid replication involving template specificity. Comparator or Comparator Group refers to sample used as the basis for comparative results.

Complex biological construct means any portion of an animal having more than one tissue type. The complex biological construct may comprise an entire limb of animal or other gross anatomical structure such as appendages, organs, collection of organs, or organ systems. The complex biological construct may include, but are not limited to, hair, bone, blood, blood vessels, muscles, connective tissue, cartilage, nerve, bone marrow, epithelium, and adipose tissues.

Dye refers to any fluorescent or non-fluorescent molecule that emits a signal upon exposure to light as apparent to those of skill in the art of molecular biology. The reporter dye refers to the dye used with the sample RNA.

Endogenous control refers to an RNA or DNA that is always present in each experimental sample. By using an endogenous messenger RNA (mRNA) target can be normalized for differences in the amount of total RNA added to each reaction. Typically, the endogenous control is a housekeeping gene required for cell maintenance such as a gene for metabolic enzyme or the ribosomal RNA.

Exogenous control refers to a characterized RNA or DNA spiked into each sample at a known concentration. An exogenous active reference is usually an in vitro construct that can be used as an internal positive control (IPC) to distinguish true target negatives from PCR inhibition. An exogenous reference can also be used to normalize for differences in efficiency of sample extraction or complementary DNA (cDNA) synthesis by reverse transcriptase.

Experiment means a group of plates analyzed together.

Gene is used to refer to a functional protein, polypeptide or peptide-encoding unit. As will be understood by those in the art, this functional term includes genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated.

Genetic molecules as referred to herein include genomic DNA, episomal DNA, messenger RNA (“mRNA”), heteronuclear RNA (“hnRNA”), transfer RNA (“tRNA”) and ribosomal RNA (“rRNA”).

Liquefaction and liquefy refer to any process in which a solid or solid suspension is homogenized so that material appears to be a liquid. The material may, in fact, be either a solution, or suspension of particles of submicroscopic size.

Multiplexing PCR means the use of more than one dye layer in an experiment and/or more than one FPR set with an associated reporter dye in each well of a plate. In one well, the target RNA and the endogenous control are amplified by different FPR sets. All the wells on a plate in an experiment will always contain the same endogenous FPR set. If there are three FPR sets used in the experiment, then all wells will have at least one of those same three FPR sets unless the wells are empty wells on the plate. Each FPR set has an associated reporter dye. A C_{T} value is reported for each FPR set in each well. A C_{T} value is recorded for each dye layer in every well on the plate.

Notebook Page means a page in a notebook used to track experiments and other confidential information.

Nucleic acid refers to DNA, RNA, single-stranded or double-stranded and any chemical modifications thereof. Modifications include, but are not limited to, those that add other chemical groups that provide additional charge, polarizability, hydrogen bonding, and electrostatic interaction.

Plate Consistency Control means a specified RNA, which is placed on every plate in multiple plate experiments to ensure consistency across plates.

Primer refers to an oligonucleotide, whether purified or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer may be single stranded for maximum efficiency in amplification but may alternatively be double stranded. If double stranded, the primer is first treated to separate its strands before being used to prepare extension products. The primer must be sufficiently long to prime the synthesis of extension products in the presence of the inducing agent. The exact lengths of the primers will depend on many factors, including temperature, source of primer and the use of the method.

Probe refers to any compound that can act upon a nucleic acid in a predetermined desirable manner, including a protein, peptide, nucleic acid, carbohydrate, lipid, polysaccharide, glycoprotein, hormone, receptor, antigen, antibody, virus, pathogen, toxic substance, substrate, metabolite, transition state analog, cofactor, inhibitor, drug, dye, nutrient, growth factor, cell. It also refers to a sequence of nucleotides, whether purified or produced synthetically, recombinantly or by PCR amplification, which is capable of hybridizing to another nucleotide sequence of interest. A probe may be single-stranded or double-stranded. Probes are useful in the detection, identification and isolation of particular gene sequences. It is contemplated that any probe used in the present invention will be labeled with a “reporter molecule,” so that is detectable in any detection system including, but not limited to, enzyme (e.g. ELISA, as well as enzyme-based histochemical assays), fluorescent, radioactive, and luminescent systems. It is not intended that the present invention be limited to any particular detection system or label.

Reference refers to a passive or active signal used to normalize experimental results. Endogenous and exogenous controls are examples of active references. Active reference means the signal is generated as a result of PCR amplification. The active reference has its own set of primers and probe.

Sample RNA or sample refers to single or double stranded RNA used in one or more experiments that may be obtained from a donor such as a person, animal or cell culture. When from an animal or person, it may be from variety of different sources, including blood, plasma, urine, semen, saliva, lymph fluid, meningeal fluid, amniotic fluid, glandular fluid, and cerebrospinal fluid, or from solutions or mixtures containing homogenized solid material, such as feces, cells, tissues, and biopsy samples. One RNA sample may be used to determine the expression of one or more genes. The same set of genes is used with every sample in the same experiment.

Standard refers to a sample of known concentration used to construct a standard curve.

Vehicle refers to substances that are injected into an animal as carriers for a test compound. Common vehicles include water, saline solutions, physiologically compatible organic compounds such as various alcohols, and other carriers well known in the art. Vehicle may also refer to a control animal injected with such a carrier in the absence of a test compound. The vehicle animal serves as a control to mimic transcriptional alterations resulting from the stress of administration but not from the drug itself.

### Calculations

As further described below in detail, the following calculations are used in connection with the subject invention:

% CV for C_{T} Values (Coefficient of Variation)=100*(StDev/Mean)

XRel (relative transcriptional change or relative expression level), This value is calculated as (1+E) (^{-ΔΔC}_{T} for FPR set), where E reflects the amplification efficiency and is assumed to be 1. E is stored as an experiment parameter and can be changed if necessary for the given experiment. XRel values greater than 1 (one) indicate more gene expression in the RNA sample than in the comparator group of the particular gene. Similarly, XRel values less than 1 (one) indicate less gene expression in the RNA sample than in the comparator group of the particular gene.

Group XRel Mean=Mean (XRel of each amplification of the FPR set in the group

Group XRel StDev=StDev (XRel of each amplification of the FPR set in the group

Group XRel SEM=StDev (XRel of each amplification of the FPR set in the group/(n)⁻⁵,

where n is the number of amplifications with FPR set in the group

% CV XREL=100*XRel SEM*SQRT (n)/XRel Mean, where n is the number of RNAs in the group.

If the amplification primers are optimized for amplification efficiency (i.e. E=1), XRel, the amount of a nucleic acid sample normalized to an endogenous reference and relative to a comparator group can be calculated by the mathematical formula:

XRel=2^{-ΔΔCT}

This above formula was derived in the following manner: The exponential amplification resulting from a given PCR reaction can be represented by the formula:

Xₙ=Xₒ×(1+E_{X})ⁿ

where Xₙ is the number of sample molecules after n cycles, X₀ is the initial number of sample molecules; Eₓ is the efficiency of sample amplification; and, n is the number of cycles.

This formula is then used to calculate the amount of product present at the threshold cycle, C_{T}. The threshold cycle is the point at which the amount of sample rises above a set threshold, typically where exponential amplification can be first detected above the background noise of the experiment. At this point, the amount of product is:

X_{T}=Xₒ×(1+E_{X})^{C}^{_{T.X}}=K_{X}

where X_{T} is the number of sample molecules at the threshold cycle, C_{T,X} is the cycle number at which the amount of sample exceeds the threshold value, and Kₓ is a constant.

In addition, a similar formula can be used to calculate the amount of amplified sample in the endogenous reference control reaction at its threshold cycle:

R_{T}=Rₒ×(1+E_{R})^{C}^{_{T.R}}=K_{R}

where R_{T} is the number of copies of the amplified endogenous reference at its threshold cycle, R₀ is the initial number of copies of the endogenous reference, E_{R} is the efficiency of amplification of the endogenous reference, C_{T,R} is the threshold cycle number for the endogenous reference, where the amplified reference exceeds the threshold value, and K_{R} is a constant for the endogenous reference.

The number of sample molecules (X_{T}) at the sample threshold cycle is then divided by the number of endogenous reference molecules at the reference threshold cycle to yield a constant designated as K:

$$\frac{{X}_{T}}{{R}_{T}} = \frac{{X}_{o} \times {( 1 + {E}_{X} )}^{{C}_{T . X}}}{{R}_{o} \times {( 1 + {E}_{R} )}^{{C}_{T . R}}} = \frac{{K}_{X}}{{K}_{R}} = K$$

The constant, K, is not necessarily equal to one because the exact values of X_{T} and R_{T} can vary for a number of reasons depending on the reporter dyes used in the probes, differential effects of probe sequences on the fluorescence of the probes, the efficiency of probe cleavage, the purity of the probes, and the setting of the fluorescence threshold.

If the amplification efficiencies of the sample and endogenous reference are assumed to be the same, i.e. E_{X}=E_{R}=E, the previous equation can be simplified to:

$$\frac{{X}_{o}}{{R}_{o}} \times {( 1 + E )}^{{C}_{T . X} - {C}_{T . R}} = K$$

which can be rewritten as:

X_{N}×(1+E)^{ΔC}^{_{T}}=K

where X_{N} is the normalized amount of sample (X₀/R₀); and ΔC_{T} is the difference in threshold cycles for the sample and reference (C_{T,X}−C_{T,R}). The equation can be rearranged as follows:

X_{N}=K×(1+E)^{-ΔC}^{_{T}}

XRel is then obtained by dividing normalized amount of sample relative to endogenous control by the normalized amount of comparator relative to endogenous control as represented by the equation:

$$\frac{{X}_{N , q}}{{X}_{N , cb}} = \frac{K \times {( 1 + E )}^{- Δ {C}_{T . q}}}{K \times {( 1 + E )}^{- Δ {C}_{T . cb}}} = {( 1 + E )}^{- ΔΔ {C}_{T}}$$

where the ΔΔC_{T}=ΔC_{T,q}−ΔC_{T,cb}. If the FPR sets are properly optimized for amplification efficiency, E should be nearly equal to one and the equation can be simplified to:

XRel=2^{-ΔΔC}_{T}

For a given experiment, as discussed in detail below, the sample RNA may be obtained from a variety of tissue sources. The sample may be animal tissue from a particular organ or animal blood or a combination. The sample might be from cell cultures. Regardless, there is typically more than one sample having the same characteristic. The common characteristic may be the type of treatment received (vehicle, compounds, etc.), the species, sex or age of the donor, or some other similar treatment. A group label is assigned to each group of samples sharing the same characteristic. Cell culture experiments in which each well on the cell culture plate is treated differently and not replicated on another cell culture plate, will result in only one sample per group. Statistical analysis assumes there is more than one sample per group and that each sample is independent of other samples treated in the same manner.

One of the groups must be identified as the comparator group. It is often the vehicle or untreated group. The comparator group may also be a particular age or time point in the experiment. The comparator group is the one group which all other groups in that experiment will be compared. For example, the comparator group may be untreated or normal sample to which the treated or diseased samples are compared. All relative expression values are defined relative to the comparator group as being either the same, higher or lower than the comparator group.

Occasionally in an experiment, there may be a need to calculate relative expression values several times using more than one comparator group. For example, it may be necessary to see the relative fold changes in a message compared to different time points in an experiment thus creating the need to easily be able to change the comparator group and quickly recalculate the relative expression values.

To run the experiment to detect RNA in the sample, the current technology of either 96 or 384 well plates may be used. C_{T} values of each well are typically supplied by the manufacturer of the polymerase chain reaction system (otherwise referred to herein as the sequence detection system). Each well may be identified as containing sample RNA or one of several types of assay controls.

There may be one or more types of control wells on each plate, or there may be no control wells. The most common type of control occurs when the experiment is performed on more than one plate, and will therefore be called the plate control. The plate controls have the same source of RNA on all the plates and are monitored to determine whether there is consistency in results across plates. The plate control may be one of the samples for which there is sufficient RNA to repeat it on all of the plates. Another type of control well is called the no template control, or NTC where no RNA is present. Thus, this control is used to determine the background signal. A third type of control well is called minus reverse transcriptase control, or -RT. These wells contain no reverse transcriptase. Thus, this control is used to check whether DNA contaminants are present in the RNA preparation.

If there are multiple plates and custom cards are not used, there should be plate controls on each plate. These RNA controls are usually matched to each gene (including endogenous) by being in the same rows or same columns as the RNA samples for that gene.

All samples and controls are replicated, having two or more wells for each sample or control. The replicates must be on the same plate and will usually be in the same row or the same column.

The RNA samples may be tested for the expression of one or more genes. The same set of genes is used with every sample in the same experiment. There will be matching endogenous control wells for each set of gene wells on the same plate that will be used in the calculations. The most common endogenous control is cyclophilin. These endogenous controls will usually be in the same rows or the same columns as the gene sample. If a sample is run for multiple genes on the same plate, the same endogenous control is used for all genes. If more than one endogenous control is present, only one will be identified for use in the calculations.

An exception occurs when custom plates are used. For example, one RNA sample may be analyzed for the transcription levels of genes plus one endogenous control. Having the endogenous control contained in the same well as the gene is called multiplexing.

Preferably, each well is specified by the following information:

1) well location on the plate or custom card,

2) sample type (unused, assay control, RNA sample, or both assay control and RNA sample),

3) group label (such as treatment group, species, sex, age, type of control, etc.),

4) sample ID (usually a number) within the group,

5) number of the FPR set(s) that identifies the gene(s).

If the sample type is assay control, the group label will identify the type of control as either plate RNA control, NTC, or -RT. The sample id field may be used to indicate the particular RNA sample corresponding to the control. For example, each RNA (sample ID) may have a -RT corresponding to it to check for DNA contamination in that sample preparation. The FPR set number identifies the gene label for the plate RNA, NTC, or -RT control results and graphs.

For the RNA samples, the sample ID may be an RNA ID from remote database or an assigned name or number. There may be two FPR set numbers for the same well, one for the endogenous control and one for the gene, if multiplexing is being performed, as in custom cards. Multiplexing can be done on regular samples or on custom plates but is not necessarily done on either.

If statistical comparisons are going to be made among groups, whenever possible, it is desirable to have the samples for the various groups on the same plate. However, it is understood that this type of plate setup is not always possible. The coefficient of variation (100 x standard deviation/mean), or CV, may be calculated for each set of replicate wells having the same group label, sample ID, and gene. The well locations of sets of wells where the CV exceeds a default value (currently 2% but may be lower), or a value that is specified by the user, are shown. The user may than choose whether to delete one or more of these wells from further processing.

The average of the replicate values is calculated for each assay control, endogenous control, and gene. A ΔC_{T} (delta C_{T}) value is calculated for each RNA sample/gene combination as the average C_{T} for the gene minus the average C_{T} for the endogenous control for that sample.

The calculations described so far can be performed at the plate level. The rest of the calculations require the data for all the plates to be available. All of the samples for the comparator group may not be on the same plate. Also, the samples for the comparator group may or may not be on the same plate as the samples for the other groups.

The median of the C_{T} values is determined for all the samples in this comparator group, regardless of plate location. Then a ΔΔ (delta delta C_{T}) value is calculated as the ΔC_{T} value for each sample minus the median (middle or average of two middle values) ΔC_{T} value for the comparator group.

As mentioned above, the relative transcriptional change (or relative expression level), XRel, is calculated as (1+E)^{(-ΔΔCT)}. E reflects the amplification efficiency and defaults to 1. Since ΔΔC_{T} will be about zero for the comparator group, its XRel value will be close to 1. XRel values greater than 1 indicate more expression than the comparator group while XRel values less than 1 indicate less expression than the comparator group by the particular gene.

There are special rules for multiplexing. When multiplexing, any given FPR set cannot exist in more than one combination of FPR sets. For example, if Gene 2 exists in a well with Gene 1 and the endogenous control (“EndoC_{T}”), then Gene 2 can ONLY exist in wells also containing Gene 1 and the endogenous control (“EndoC_{T}”). Gene 2 cannot exist in a well of any other combination. For example, Gene 2 cannot exist in a well containing Gene 3 and the EndoC_{T}. Any multiplexing experiment not following this rule will result in the reporting of invalid calculations.

The present invention is suitable for any two-dimensional plate configuration including but not limited to 96-well plates, 384-well plates, custom or standardized. The invention has the capability to analyze data from a partial plate, single plate or multi-plate experiment. Single dye or multiple dye (multiplexed) analysis can also be accommodated. The computer readable program code will accept any plate layout, unlimited number of RNA samples and unlimited number of primer/probe sets (FPR sets) in an experiment. Exported result files from any experiment run can be loaded into the program for calculation.

As part of the subject invention, the user may choose which FPR set is treated as the endogenous control and which RNA group is treated as the comparator group, making it possible to compare reports with different endogenous control and comparator group combinations. In addition, percent CV (% CV) between replicate wells on a plate may be calculated and outlier replicates are flagged. The mean, standard deviation, and standard error of the mean among RNA groups may also be calculated.

As further described below, experiment analysis involves a series of steps. The results of the analysis may be displayed in a Microsoft excel workbook and the like or on an information-display apparatus. There are various levels of documentation and display of information. From the PCR system, typically a printout or other display is obtained that shows the details of the C_{T} values for each well on the plate. The present invention calculates and displays from these values should show, by gene, the C_{T}, ΔC_{T}, ΔΔC_{T}, and XRel values for each sample in each group.

In addition, a summary may be shown for each group and gene that contains the descriptive statistics for the group (n, mean, and standard error of the mean). A graph may be produced for each gene that displays the group means (with error bars). Furthermore, an electronic output file should be generated that contains the XRel values for each sample along with the gene label, group label, and sample id. This output file can then be used for further statistical analysis.

More detailed database files may be produced using the original plate reader values so that, if desired, the calculations may be re-done, exercising different options. Graphics may be produced for assay validation purposes. Assuming that the C_{T} values are available on the same plate for endogenous control samples and assay controls, a graph is produced whose X-axis may display the C_{T} average of the endogenous control wells that were used to calculate ΔC_{T} for all of the genes on the plate. The Y-axis may then display the C_{T} values for the various types of assay control wells, by gene, including endogenous. The symbol printed reflects the gene label, as described in a legend. There may be as many of each symbol as there are plates in the assay.

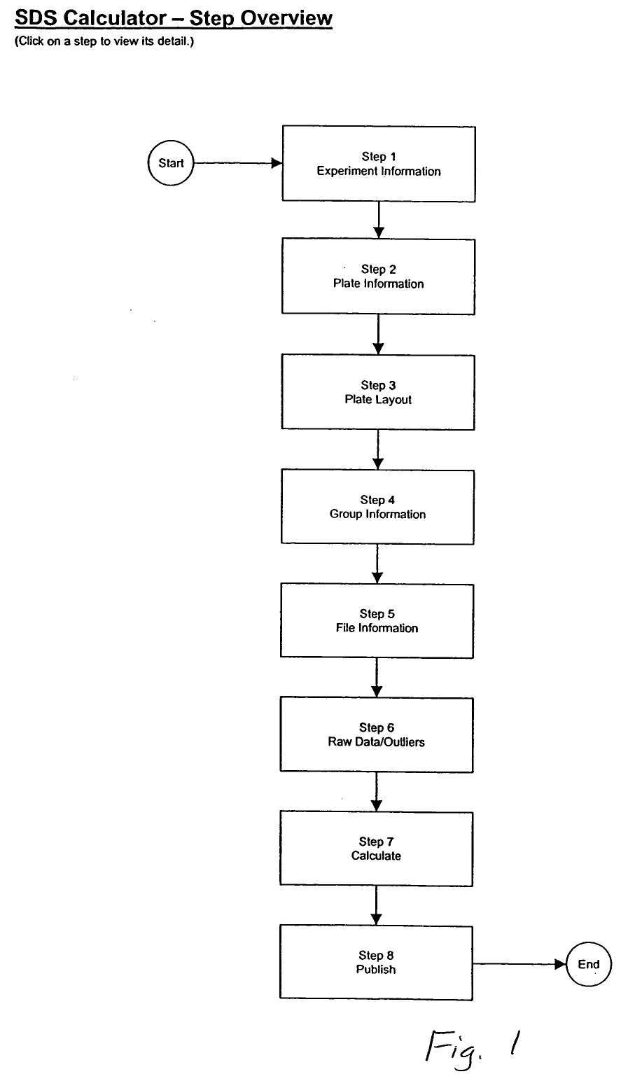

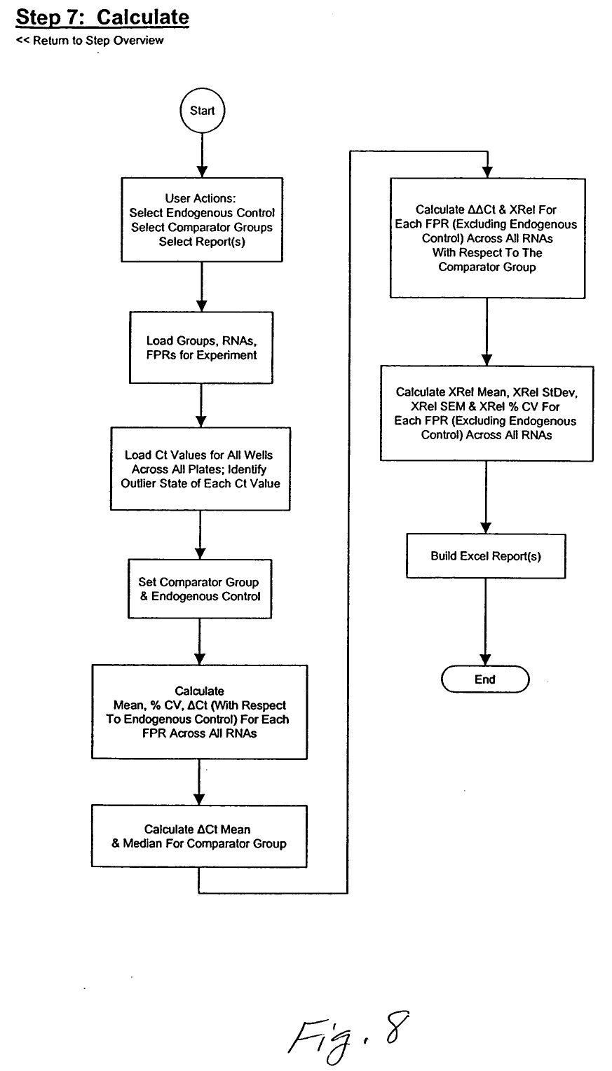

If assay control samples are not available, a bar chart of endogenous control wells may be provided. The bar for each plate reflects the mean, while the error bars reflect the minimum and maximum C_{T} values. Similar tables and graphs may also be produced for NTC and -RT controls. As shown in FIGS. 1 to 9, the method of the subject invention comprises a number of specific steps. FIG. 1 depicts the overall methodology of present invention.

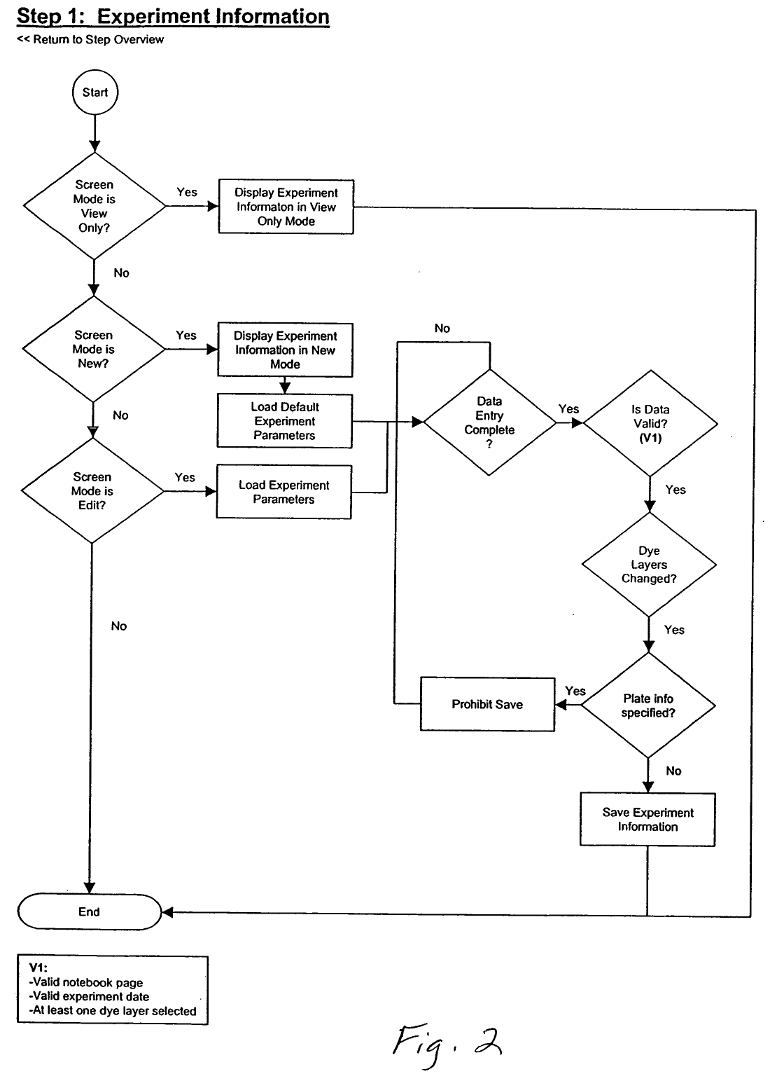

FIGS. 1 through 9 are logic flow diagrams depicting the overall methodology used in a computer system of the present invention for analyzing an experiment to detect RNA. FIG. 2 is a flow chart of the first step, the recording of experiment information. To create a new experiment, a separate screen is displayed and information provided such as experiment ID, description, dye layers and other parameters including notebook page reference, outlier cutoff, and amplification efficiency “E.” Experiments can also be deleted from the database. However, it is recommended that a privilege be attached to this function.

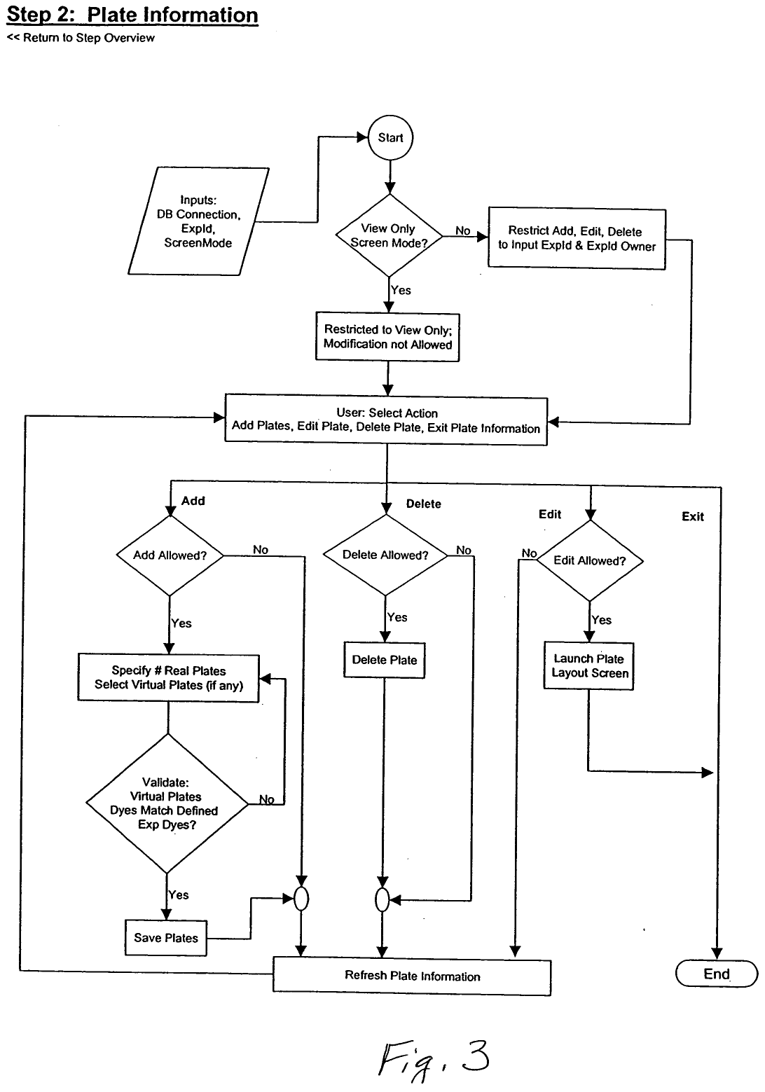

In the step 2, plate information including the number of plates and type of plates are specified. FIG. 3 is a flow chart of this second step. Real or virtual plates may be specified. A virtual plate may be a plate from a previous experiment. Plates of varying size may be selected. For a new experiment there are initially no plates defined. Plates may be added to the experiment in an unlimited number as real or virtual plates.

Real plates are the new plates defined for the current experiment. Data files gathered at the time of the experiment shall be parsed and recorded under the appropriate plate. A type of plate is also chosen such as 96 well or 384 well plate or custom card. Virtual plates are plates that already exist on another experiment. The data for these plates was gathered on the other experiment. Virtual plates are optional

For example, the first experiment is at time zero, the second experiment is at time 3 months, and the third and current experiment at time 6 months. The analysis for this current experiment would include the plate date from the previous two experiments, time zero and time 3 months. The current experiment, time 6 months, would then include its own plates (real plates), along with the plates from the previous two experiments, time zero and time 3 months, as virtual plates. When adding virtual plates to an experiment, the dyes used on the virtual plate must match the dyes for the experiment. For example, an experiment defined as using the FAM dye cannot have a virtual plate using the VIC dye.

When specifying plate information, information about the particular plate is included such as number of wells, well type, dye layers, and FPR set. The contents of the well or well type may be minus RT, plate consistency control, sample, and sample and plate consistency control. Each well either contains RNA or is NTC. All wells that are not empty contain an FPR set.

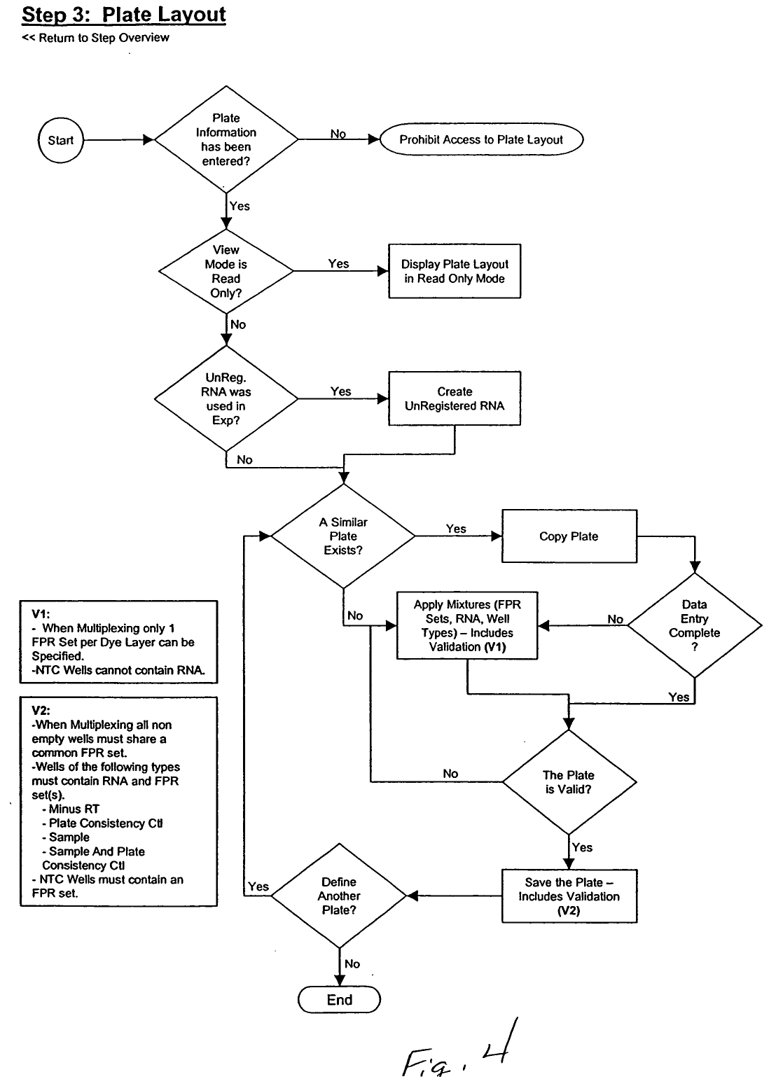

In Step 3, and as shown in FIG. 4, the plate layout including defining FPR sets and RNA associated to each well on the plate for the experiment is provided. Prior to generating this information, both experiment information and plate information must have been completed. The FPR sets are categorized by dye layer and species. To apply an FPR set, select the wells of interest and select the desired FPR set. Conversely, the remove an FPR set, select the wells of interest and delete or remove the FPR set from its designation.

If the experiment is multiplexed, only one FPR set per each dye layer may be used in each well. Dye layers are associated to the experiment through the experiment information. If the experiment is not multiplexed, only one FPR set per well can be specified. When applying FPR sets, if any of the selected wells already contain an FPR set they will not be overridden with the FPR set that is currently selected. To replace an FPR set, the existing FPR set must be removed or deleted first.

RNA is categorized by the user and once recorded as part of an internal database is referred to as registered. When the user changes, the relevant registered RNAs are listed. To apply registered RNA, select the wells of interest and the registered RNA. To remove registered RNA, select the wells of interest and delete the registered RNA. Only one registered RNA per well may be specified. Whey applying registered RNA, if any of the selected wells already contain registered RNA, they will be overridden with registered RNA that is currently selected. To replace registered RNA, it must be removed. Registered RNA cannot be applied to NTC wells or empty wells.

To create unregistered RNA or RNA that has not been previously recorded, identify the number of unregistered RNA to generate. At this time, the name, notebook page and comments may be associated to the unregistered RNA. This unregistered RNA information may be modified if necessary. The unregistered RNA is then associated with wells of interest. Only one unregistered RNA may be specified per well. Unregistered RNA will not be applied to wells already containing unregistered RNA. The unregistered RNA must be removed from a well prior to selecting another unregistered RNA. Unregistered RNA may not be applied to NTC wells or empty wells.

A number of various well types are available for use in connection with the method of the subject invention. The types of wells include, but are not limited to, the following: sample, NTC, RT, plate consistency, sample and plate consistency, or empty. Plate information including FPR sets, registered RNA, unregistered RNA, and well type may be copied from another plate. In order to save plate information, wells of the following types must contain RNA and FPR sets: Minus RT, Plate consistency control, sample, and sample and plate consistency control. NTC wells must contain an FPR set. When multiplexing, all non-empty wells must share a common FPR set.

The next step (step 4) in the method of the subject invention is to create and populate RNA groups. FIG. 5 is a flow chart of this step. An RNA group can be only one RNA but may contain multiple RNAs. Both registered and unregistered RNA are available to assign to groups. Only RNA belonging to a sample or sample and plate consistency wells is provided here. Each new RNA group shall have a group name. Each specific RNA is assigned to a group and may be later removed if necessary. All RNA must be assigned to at least one group.

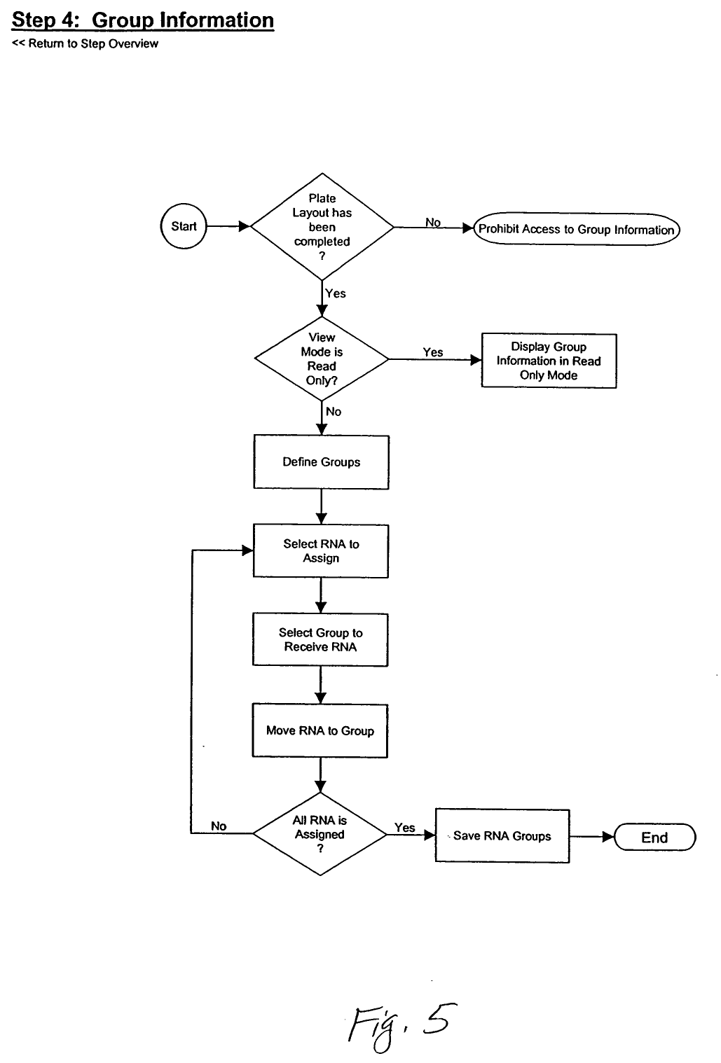

In step 5, exported data files are associated to specific real plates in the experiment. As shown in FIG. 6, the file information for virtual plates used in the experiment already exists and may be overwritten. Any one of a number of data file formats may be utilized. If an endogenous control was not specified, an endogenous control gene must be selected at this time.

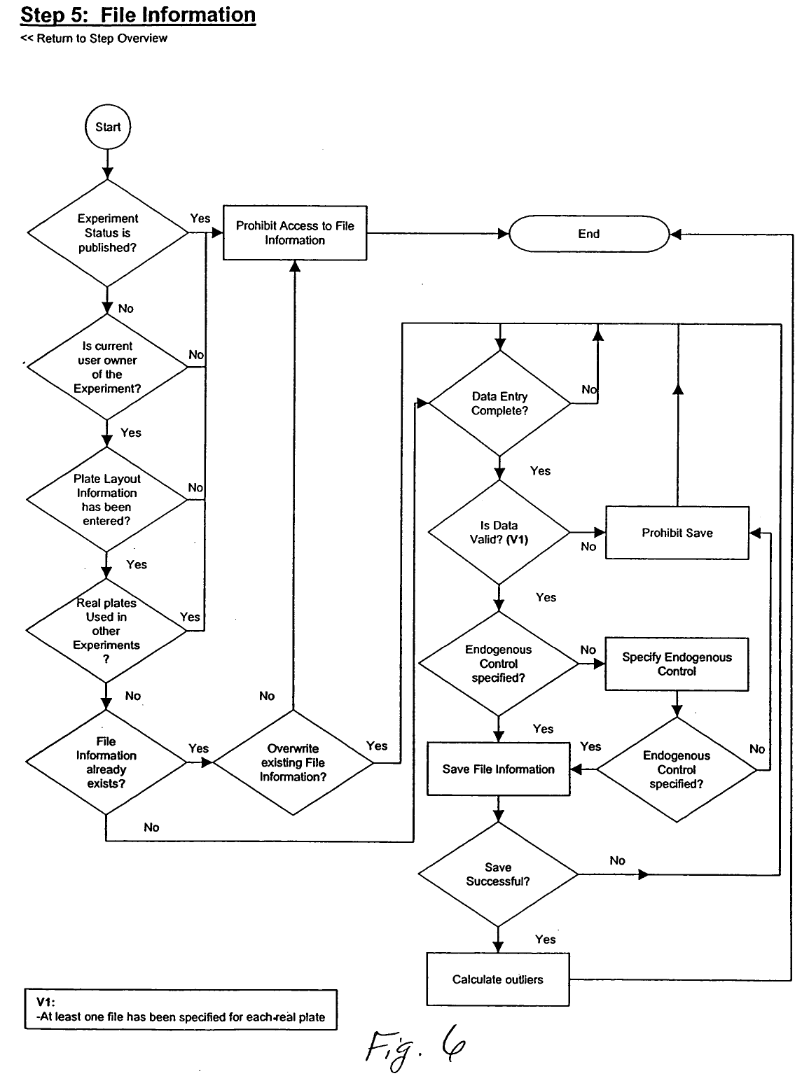

In step 6, C_{T} values may be reviewed and outliers managed. Outliers may be calculated at any point, up to the time the experiment has been published. As shown in FIG. 7, outliers may be turned on or off at the well level for each dye layer. Two types of outliers exist including auto outliers identified during the file information step and user outliers explicitly set by the user. Several outlier values may be identified at one time. When multiplexing, outliers may be viewed for different dye layers. Once all dye layers have been accessed, outliers may be saved or recalculated.

Outliers are determined by calculating the coefficient of variation, CV, for each set of replicate Ct values within the same RNA Group. A replicate Ct value is defined as a sample well containing the same FPR Set and the same RNA. When multiplexing, a sample well may contain multiple Ct values. If the CV for a replicate Ct value exceeds a predetermined percentage, that Ct value is marked as a auto outlier. Marking a Ct value as an auto outlier indicates that the user should review that Ct value for accuracy. If the user determines that the Ct value should not be included in any calculations, the user has the ability to mark it as a user outlier. Marking a Ct value as a user outlier prevents that value from being used in any calculations.

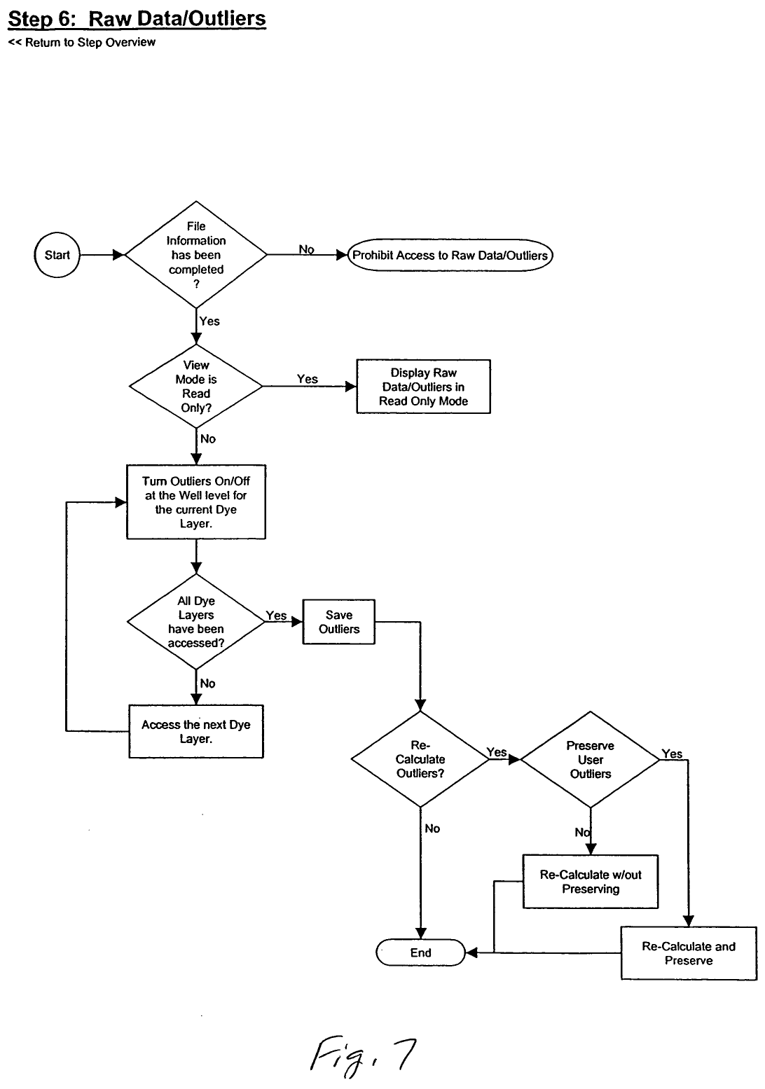

In step 7, as shown in FIG. 8, the calculations are completed. First, the endogenous control and comparative groups are selected. The endogenous control and comparative groups are the basis behind the reported calculation for all genes. Choosing different comparative groups is a unique feature of the method of the subject invention. Through this feature it is possible to compare delta delta C_{T} and XRel results with different comparative groups. The user may exclude marked outliers if necessary.

The endogenous control is initially selected by the user at the time data are parsed for the experiment (step 5 described above). The auto outlier process is performed any time data are changed in experiment analysis. The user may select a different endogenous control during the calculations (step 7) of the analysis. If the endogenous control is changed, the user may run the outlier process again to reflect a change in the endogenous control.

In order to determine the relative expression value of any given sample, one sample (RNA) or group of samples (group of RNAs) must be chosen as a comparator. The comparator group is one to which all other groups will be compared. All relative expression values are defined relative to the comparator group as being the same, higher or lower than the comparator group.

Occasionally in an experiment, there may be a need to calculate relative expression values several times using more than one comparator group. For example, it may be necessary to see relative fold changes in a message compared to different points in the experiment thus creating the need to easily be able to change the comparator group and quickly recalculate the relative expression values.

The ability to choose different comparator groups is a feature of the subject invention that makes it possible to compare ΔΔC_{T} and XREL results using different comparator groups.

Calculations are made with respect to each endogenous control for each FPR set across all RNAs for the following: mean, % CV and delta C_{T}. Calculations for each comparator group include delta C_{T} mean and median. Across all RNAs with respect to the comparator group the delta delta C_{T} and XRel for each FPR set is calculated. XRel Mean, XRel standard deviation, XRel SEM and XRel % CV is calculated for each FPR set across all RNAs excluding endogenous control. The subject invention is a method and apparatus for the extraction and isolation of genetic molecules such as DNA, RNA, mRNA, rRNA or tRNA from an animal for use in the analysis of genetic expression. The present method and apparatus of the subject invention are particularly useful in high throughput, automated analysis of genetic molecular levels and function.

To extract and isolate genetic molecules for use in the above analysis, the subject invention further includes a method of extraction and isolation of genetic expression that comprises the steps of liquefying or pulverizing a complex biological construct into solution or powder having complete and uncontaminated genetic molecules, transferring the solution to a Taqman assay or microarray, and determining gene expression and/or function. The apparatus for performing the method comprises a chamber fitted with a component that will fracture the complex biological construct and ruptures it cells. The apparatus also comprises a means for applying mechanical force to the chamber whereby the component will rupture the cells releasing genetic molecules into solution.

A complex biological construct useful in the method of the present invention may contain many of the tissues that make up an animal. The body of the animal, also referred to as the organism, can be understood at seven related structural levels: chemical, organelle, cellular, tissue, organ, organ system and finally the entire body or organism, or a discrete portion or part of it. A tissue by definition is a group of cells with similar structure and function. An organ is composed of two or more tissue types that perform one or more common function. The organ system is a group of organs classified as a unit because of a common function or set of functions. The complex biological construct of the subject invention, however, will contain several types of tissue potentially having a diversity of function and may potentially contain numerous cell types. For example, there are over 200 types of cells in the human body assembled into a variety of tissue types.

The four primary tissue types are epithelial, connective, muscular, and nerve. Each primary tissue type has several subtypes. Epithelial tissues include membranous and glandular. Connective tissues include connective tissue proper and specialized connective tissue. The three subtypes of muscle tissue are skeletal, cardiac and smooth. The nerve cells are specialized form of communication and are composed of a network of neurons among supporting glial cells. The epithelia and connective tissues are the most abundant and diverse of the four tissue types and are components of every organ in the human body.

In epithelial tissues, cells are tightly bound together into sheets called epithelia. The epithelia tissue consists primarily of cells, and it is cells rather than the matrix that bear most of the mechanical stress. Epithelial cell sheets line all the cavities and free surfaces of the body and the specialized junctions between the cells enable these sheets to form barriers to the movement of water, solutes, and cells from one body compartment to another. Epithelial sheets almost always rest on a supporting bed of connective tissue which may attach them to other tissues such as muscle that do not themselves have either strictly epithelial or strictly connective tissue organizations.

There are many specialized types of epithelia. However, whereas epithelia may be specialized for unique functions in an organ system, they all have some features in common. First, the cells are apposed to one another and line a surface. Second, they sit on a layer of fine filaments, called a “basal lamina”. Collectively these layers form a boundary between the external environment and the remainder of the organ. Thus, at the most basic level, epithelia are organized to control movement of substances into and out of that organ.

In addition, a stratified epithelium may provide more protection to the organ against friction and the like since the outer layers of the cells could be sloughed off as the epithelium encounters friction. Simple epithelia regulate transport through the epithelial cells by membrane transport proteins, endocytosis and special barrier junctions.

The shape of the cell facilitates determination of its function. For example, flattened, scale-like cells (referred to as squamous) may be seen in one layer (simple) or in multiple layers (stratified). If these cells are in a single layer, they provide minimal protection, but often provide more opportunity for passive transport of substances across the cell. For example, the capillary wall is where epithelial cells provide the surface area for transport of gases and other molecules. If squamous cells are in a stratified epithelium, they are often designed for protection against invasion or friction. They have desmosomes junctions) and can be sloughed off and replaced rapidly.

Epithelia that are cube shaped are called, appropriately, “cuboidal”. Often these epithelia have specialized junctions and transport processes that control movement of substances from one side to the other. Sometimes they are secretory. Thus, the taller the cell, the more active it may be in terms of regulated transport. This is particularly true of the tallest epithelial cells, the columnar cells. Shaped like a column, these cells often have very different, specialized surfaces designed to protect the barrier and transport into the cell and then out of the cell. Some epithelial cells, such as the thyroid, become taller as they secrete more.

Finally, there are the transitional epithelium in bladder or ureter that are not classified. This epithelium may have cells that are squamous and even columnar. It is definitely multilayered. It also may distend so that it looks like it is only 2-3 cellular layers.

Various types of cells in the epithelium perform different function. Absorptive cells in epithelial have numerous hair-like microvilli projecting from their free surface to increase the area for adsorption. Ciliated cells have cilia in their free surface that beat in synchrony to move substances over epithelial sheet. Secretory cells are found in most epithelial layers and exude substances onto the surface of the cell sheet.

Connective tissues are classified as connective tissue proper and specialized connective tissue. The specialized connective tissue includes cartilage, bone, and blood. Connective tissue proper has a matrix comprising numerous fibers that are collagenous, elastic, or reticular (branched). The connective tissue proper includes dense connective tissue and loose connective tissue. The loose or areolar connective tissue has an intercellular matrix widely distributed in the body and found most readily beneath the skin and superficial fascia (fatty connective tissue) separating muscles, in all potential spaces, and beneath the epithelial lining in lamina propria of the digestive system. The web-like tissue binds cells and organs together but permits the cells and organs to move, as necessary in relation to each other. Loose connective tissue is composed of a large amount of amorphous ground substance whose consistency varies from liquid to gel, allowing cells to move around freely and other structures such as blood vessels and nerve, to pass through it. This type of connective tissue is important because of its cellular content in the defense against infection and the repair of damaged tissues.

Cells found in the loose connective tissue include, but are not limited to, the following: fibroblasts, which synthesize collagenous connective tissue fibers that are flexible but of great tensile strength; macrophages and monocytes, which ingest, digest, or collect microscopic particles such as debris of dead cells; certain microorganisms; and other non-biodegradable matter. Mast cells synthesize and release substances of physiological importance (e.g., heparin and histamine).

Dense connective tissue appears in two forms: dense irregular and dense regular connective tissue. The irregular type is found in the dermis of the skin, deep fascia surrounding and defining muscles, capsules of organs and nerve sheaths. Dense regular connective tissue is found primarily in ligaments and tendons and also in ligaments, aponeuroses and the cornea of the eye. While a tendon may be confused with striated muscle at low magnification, the structural differences are easily apparent at higher magnifications. Dense connective tissue contains fewer cells, but, when present, the cells are similar in type to those found in loose connective tissue. Collagenous fibers predominate in dense connective tissue.

Cartilage is a non-vascular tissue containing fibrous connective tissue (collagen Type 2) embedded in an abundant and firm matrix. The cells that produce cartilage are called chondroblasts, and, in mature cartilage where the cells are housed in lacunae, they are termed chondrocytes. Three types of cartilage are recognized: hyaline, elastic, and fibrocartilage. Hyaline cartilage is found at the ventral ends of ribs and in the nose, larynx, trachea, and articular surfaces of adjacent bones of movable joints.

Fibrocartilage is composed predominantly of collagenous (Type 1) fibers arranged in bundles, with cartilage cells surrounded by a sparse cartilage matrix between the fibrous bundles. Fibrocartilage has characteristics similar to both dense connective tissue and hyaline cartilage. It is always associated with dense connective tissue, and, because of its usual paucity of cartilage cells, there appears to be a gradual transition between the two types of connective tissue. Although cartilage cells are not abundant, they are arranged in scattered clusters in parallel arrays, reflecting the direction of stresses placed upon the tissue. Fibrocartilage has no identifiable perichondrium and differs in this regard from hyaline and elastic cartilage. Elastic cartilage is found in the external ear (pinna), auditory tube, epiglottis, and corniculate and cuneiform cartilages of the larynx.

Bone is a tissue that forms the greatest part of the skeleton and is one of the hardest structures of the body. It is the rack upon which all the soft parts are suspended or attached. The skeleton is tough and slightly elastic, withstanding tension and compression. Bone differs from cartilage by having its collagenous connective tissue matrix impregnated with organic salts (primarily calcium phosphate and lesser amounts of calcium carbonate, calcium fluoride, magnesium phosphate, and sodium chloride). The osteoblasts, which form the osseous tissue, become encapsulated in lacunae but maintain contact with the vascular system via microscopic canaliculi. When encapsulated, they are referred to as osteocytes.

Blood and lymph is a type of connective tissue that is peculiar because its matrix is liquid. The blood is carried in blood vessels and is moved throughout the body by the contractile power of the heart. Lymph is found in lymph vessels but originates in extracellular spaces as extracellular fluid, which is normally extravasated from blood capillaries. The extracellular fluid, which enters the lymphatic system of vessels, will have mononuclear white blood cells added to it as the fluid is filtered through lymph nodes, which produce such cells. Lymph is returned to the blood stream near the right and left venous angles (junction of the internal jugular and subclavian veins).

Derived from embryonic mesoderm, mesenchyme is the first connective tissue formed. The cells are widely spaced, with an abundance of intercellular matrix. The primitive mesenchymal cells differentiate into all the supporting tissues of the body. The cells derived from the mesenchyme include blood cells, megakaryocytes, endothelium, mesothelium, reticular cells, fibroblasts, mast cells, plasma cells, special phagocytic cells of the spleen and liver, cartilage cells, and bone cells as well as smooth muscle.

Widely distributed in the embryo as a loose connective tissue, mucoid tissue is composed of large stellate fibroblasts in an abundant intercellular substance, which is homogeneous and soft. In the umbilical cord, it is known as Wharton's jelly.

Muscle cells produce mechanical force by their contraction. In vertebrates there are three main types of muscle. Skeletal muscle moves joints by its strong and rapid contraction. Each muscle is a bundle of muscle fibers, each of which is an enormous multinucleated cell. Smooth muscle is present in digestive tract, bladder, arteries, and veins. It is composed of thin elongated cells (not striated), each of which has one nucleus. Cardiac muscle, intermediate in character between skeletal and smooth muscle, produces the heartbeat. Adjacent cells are linked by electrically conducting junctions that cause the cells to contract in synchrony.

Nerve tissue is specialized tissue making up the central and peripheral nervous systems. Nerve tissue consists of neurons with their processes, other specialized or supporting cells such as the neuroglia, and the extracellular material.

Neuroglia is the supporting structure of nerve tissue. It consists of a fine web of tissue made up of modified ectodermal elements, in which are enclosed peculiar branched cells known as neuroglial cells or glial cells. The neuroglial cells are of three types: astrocytes and oligodendrocytes (astroglia and oligodendroglia), which appear to play a role in myelin formation, transport of material to neurons, and maintenance of the ionic environment of neurons; and microcytes (microglia), which phagocytize waste products of nerve tissue.

The complex biological construct of the subject invention contains at least two subtypes of tissue, each having a different function. The tissues of the complex biological function have diverse function. For example, the complex biological construct may be the paw of an animal having muscle, bones, nerves, skin, connective tissue and hair. In another example, the complex biological construct may be the entire digestive tract of an animal including, but not limited to, muscle tissues from the walls of the stomach and intestine, tissue producing digestive enzymes, and the microvilli of the intestine involved in nutrient absorption.

Isolation of a complex biological construct employs any method of separating and/or severing the construct from an animal. The isolation may be done by surgical procedures on an anesthetized animal including surgical extraction or resection and amputations. Methods resulting in termination of the animal include dissection, severing and excision.

In the preferred embodiment, the complex biological construct is flash frozen with liquid nitrogen immediately after euthanization to maintain the subcellular contents of the construct in the same state as at the time of isolation. Subcellular components include any molecule, macromolecule, or structure present originally within the cell or on the cell surface or which results from the breakage of the cells. Examples include nucleic acids, proteins, metabolites, macromolecular complexes, and desmosomes. Specific proteins may include enzymes, structural proteins, receptors, and signaling proteins. Macromolecular complexes include ribosomes, cytoskeletal fragments, chromosomes, proteosomes, and centromeres.

Flash freezing may be any method where the complex biological construct is completely frozen intact or as a solution or suspension of subcellular components within a few seconds after exposure to cold temperatures. This is generally accomplished by applying extreme cold to the subject via a cryogenic liquid such as liquid nitrogen or dry ice suspended in an alcohol.

Complex biological constructs are tested based on their role in a disease process or their role in a normal function. Problems may arise if only a few cells in the test construct are actively involved in the mechanism or event. Hence, the remaining cells can dilute any signal that could be detected by physical mass alone. For example, 1% of the cells in a tissue give a signal but the remaining 99% mass dilutes the signal to less detectable or nondetectable.

The complex biological construct is then liquefied in lysis buffer (either alone or in combination with a lysis buffer). When the complex biological construct is liquefied, cell lysis occurs. Cell lysis is the rupturing of the cell's plasma membrane and ultimately resulting in the death of the cell. When the cell's plasma membrane is ruptured, the contents of the cell are released. Cell content includes: endoplasmic reticulum responsible for the synthesis and transport of lipids and membrane proteins; mitochondria; cytosol; Golgi apparatus; filamentous cytoskeleton; lysosomes or membrane-bounded vesicles that contain hydrolytic enzymes involved in intracellular digestions; peroxisomes or membrane-bounded vesicles containing oxidative enzymes that generate and destroy hydrogen peroxide; and the cell nucleus.

The cell nucleus stores genes on chromosomes, organizes genes into chromosomes to allow cell division, transports regulatory factors and gene products via nuclear pores, produces messenger ribonucleic acid (mRNA) and organizes the uncoiling of DNA to replicate key genes. The cell nucleus is separated from the cytoplasm by the nuclear envelope. The nuclear contents communicate with the cytosol by means of openings in the nuclear envelope called nuclear pores. The nucleus also has the nucleolus where ribosomes are produced. The nucleolus is organized from the nucleolar organizing regions on different chromosomes. A number of chromosomes transcribe ribosomal RNA at this site.

All of the chromosomal DNA is held in the nucleus, packed into chromatin fibers by its association with histone proteins. Before cell division, the DNA in the chromosomes replicates so each daughter cell has an identical set of chromosome. DNA is responsible for coding all proteins. Each amino acid of DNA is designated by one or more set of triplet nucleotides, code produced from one strand of DNA, by a process called transcription, producing mRNA. mRNA is sent out of the nucleus where its message is translated into proteins. Translation may be done in the cytoplasm on clusters of ribosomes called polyribosomes or on the membranes of the endoplasmic reticulum. The ribosomes provide the structural site where the mRNA sits. The amino acids for the proteins are carried to this site by transfer RNA (tRNA). Each tRNA having a nucleotide triplet that binds to the complementary sequence on the mRNA.

A lysis buffer is a solution containing various components that facilitate cell lysis or cell rupture, and stabilize resulting intracellular components. Examples include detergents, salts, nuclease inhibitors, protease inhibitors, metal chelators such as EDTA and EGTA, lysozyme, and solvents.

The method of the subject invention is especially useful for the extraction and isolation of genetic molecules such as DNA or RNA. The use of a complex biological construct as opposed to a particular tissue sample or organ eliminates the need to analyze the expression patterns in each and every tissue therein to gain an understanding of gene expression patterns within the construct.

In one preferred embodiment of the present invention, the frozen complex biological construct is placed into a sealed chamber along with a liquefying or pulverizing component (herein sometimes referred to as “component”). By the application of force, the liquefying or pulverizing component will disrupt, breakdown and break up the complex biological construct.

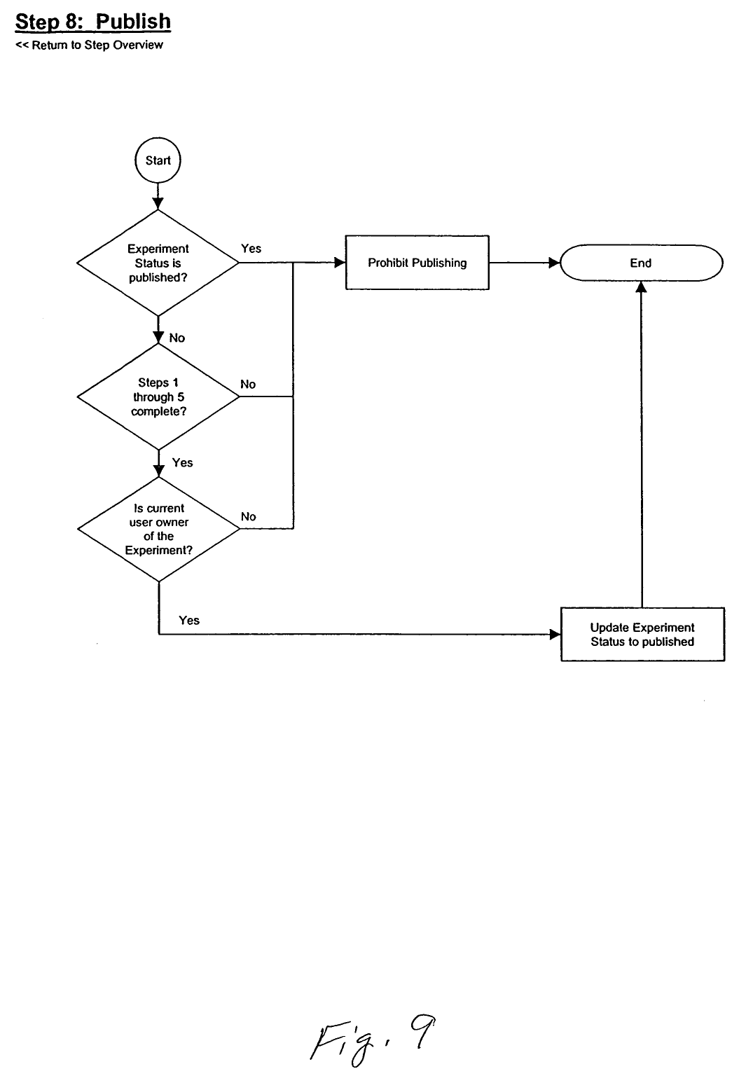

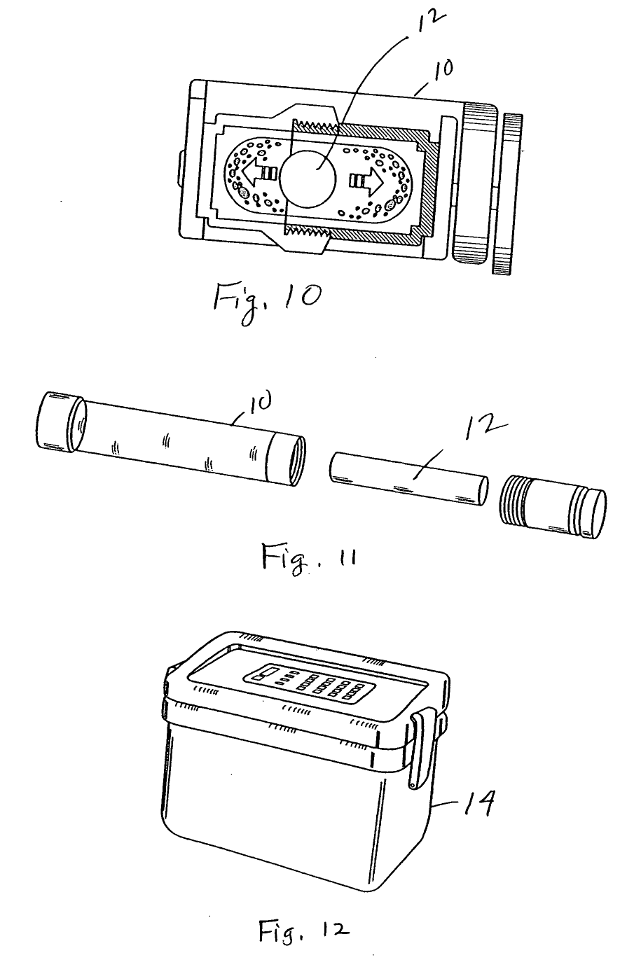

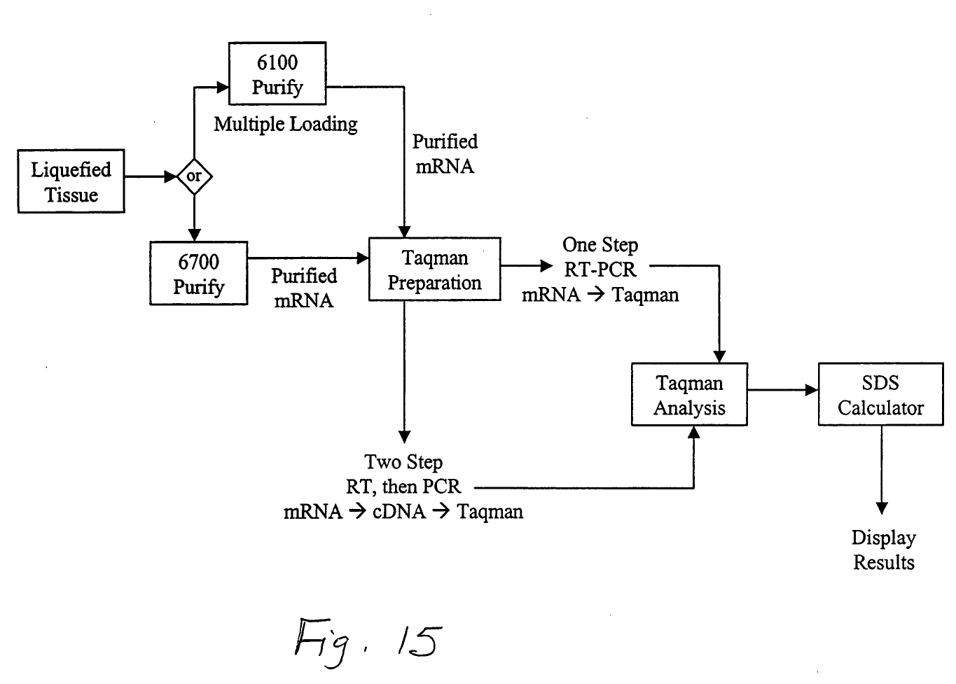

As shown in FIGS. 10 through 14, the apparatus of the preferred embodiment includes a chamber 10 suitable for containing the biological construct and pulverizing or liquefying component 12. The chamber 10 refers to any container designed to hold a complex biological construct. Preferably, the chamber 10 will be of constant shape and diameter in two dimensions to facilitate movement of the component throughout the entire chamber. The chamber 10 may be in the shape of a tube or cylinder, either straight or curved. Preferably, the interior of the chamber 10 will be made of the same material as the component 12 to prevent excessive wear of either the chamber or component 12 from contact of surfaces of varying hardness. The chamber 10 may be made of stainless steel, porcelain glass, chrome steel, agate, or any other appropriate material. Preferably, the interior of the chamber 10 will be made of stainless steel, or, in the case of the freezer mill 14, may be plastic with steel ends.

Suitable chambers include microtube containing small beads, cylinder with closely fitting beads or impactors such as the large cylindrical chamber produce by Retch®, the cryogenic tube-like chambers of the SPEX® CertiPrep 6750 Freezer/Mill 14, and spherical or hemispherical chambers such as that BioSpec® Beadbeater®.

The chamber 10 is designed to facilitate the movement of the liquefying or pulverizing component 12 (as referred to sometimes as a grinding element 12) in and through the chamber 10, or in the case of the freezer mill 14, the tissue moving through a magnetic field which in conjunction with a stainless steel rod within the cylinder powders the tissue. This component 12 may be any object that applies mechanical force or abrasion to the contents of the chamber 10. The component may be a sphere, piston, cylinder closely fitted to the contours of the chamber described above. Alternatively, the component 12 may consist of small beads or sand, a hammer, an abrading surface, or any object capable of crushing, smashing, striking, abrading, compacting, or otherwise bearing on an object.

The component 12 may be considerably smaller than the chamber 10 and thereby capable of free movement therein. Alternatively, the component 12 and chamber 10 may be designed so that the component 12 is shape and size to a cross section of the chamber 10, which is held constant along the length of the chamber 10, thereby allowing lateral movement of the component 12 back and forth across the chamber 10.

A mechanical assembly is provided for imparting motion to either the component 12 or the chamber 10. In a preferred embodiment, the chamber 10 is oscillated, imparting momentum to one or more freely moveable components present therein.

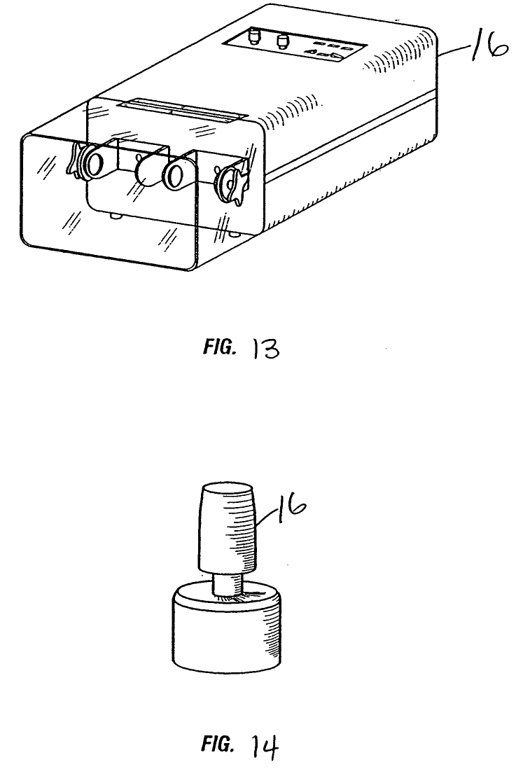

An assembly may be any mechanical device capable of being placed in motion, either manually or by a motor. FIGS. 12 and 13 depict two examples of such assemblies. The assembly may take the form of a mechanical arm, platform, centrifugal device, and magnetically driven impacting devices such as pistons and beads. Oscillatory motion and oscillation refer to any motion that follows a repetitive pattern. Said motion may consist of vibrations, shaking, rocking or swinging. This oscillation may be driven either by applying motion to the grinding element or the assembly itself.

Mechanical force may be applied to the chamber itself to impart momentum to a freely mobile component 12 within the chamber 10, or to the component 12. High speed physical impact of the component 12 on the complex biological construct will result liquefaction or pulverization of the construct, rupture of the cells, and release of intracellular components from the construct.

Devices are currently available in which biological samples are processed into intracellular component through the rapid oscillatory motion of beads, spheres or other objects through a sealed chamber containing the sample. These include the SPEX® CertiPrep 6750 Freezer/Mill, the BioSpec® Beadbeater®, the Retsch® Mixer Mill MM 300, and the Qiagen® Mixer Mill MM 200 (see e.g. FIGS. 3 and 4). Also, as shown in FIG. 5, any type of tissue crusher 18 may be utilized to process the biological sample.

As shown in FIG. 12, the SPEX® CertiPrep 6750 is designed to grind a wide variety of samples including polymers, wood, rubber, and biological tissues. The grinding is carried out at cryogenic temperatures, which provides the advantages of increasing the brittleness of the sample and preventing heat degradation during the grinding process. The grinding itself is vibratory movement of magnetically driven steel impactors through one to four individual grinding chambers. Each grinding chamber 10 or vial is composed of either a polycarbonate or a stainless steel central section with steel endplugs that can withstand the impact of the grinding elements. A magnetic coil drives the motions of the steel impactor and is placed around the chamber. Cryogenic temperatures are maintained by immersing the chambers and coils in liquid nitrogen during the liquefying pulverization since this is only grinding process.

The BioSpec® Beadbeater® is specifically designed for cell disruption. A solid Teflon impeller rotating at high speed forces thousands of minute glass beads to collide with the sample in a specially designed chamber. 90% disruption of the cells can be achieved in less than three minutes.

As shown in FIG. 13, the Retsch® mixer mill 16 is designed as all-purpose grinder capable of processing a large variety of samples ranging from minerals and ores to biological cells. The sample is placed in specially designed chambers made out of a variety of materials including stainless steel, agate, hard porcelain, tungsten carbide, zirconia, and Teflon® along with one or more specially designed balls made out of similar materials. Rapid vibration of the chamber at vibrational frequencies as high as 60 Hz propel the balls through the chamber 10. The disadvantages of the Retsch® mixer mill 16 are it's reliance on the specially designed chambers and the fact that it can only process two chambers at one time if large masses of tissue are used. Forty-eight small tissue samples (2 mg-20 mg) can be processed if an adaptor is used. The Qiagen® mixer mill functions very similarly to Retsch® system but is only designed for the processing of biological samples. The Qiagen® system offers the advantage of being able to process up to 192 samples at the same time using special adaptors that can hold either 96 1.2 ml microtubes or 24 1.5-2.0 ml microtubes. The Qiagen® mixer mill can also process larger sample volumes using the chambers manufactured by Retsch® but like the Retsch® system cannot accommodate more than two such chambers at a time. Qiagen® 3 mm tungsten carbide beads for processing of the smaller samples but similar stainless steel beads can be obtained from either Retsch® or BioSpec®. Like the Retsche mixer mill, the beads are propelled by rapid vibration of the chamber or tubes, which can be carried out at 3-30 Hz vibrational frequency.

FIGS. 15 through 20 depict an overview of a typical high throughput RNA laboratory. FIG. 15 is a logic flow diagram of the laboratory depicting the individual processes required to analyze the RNA sample.

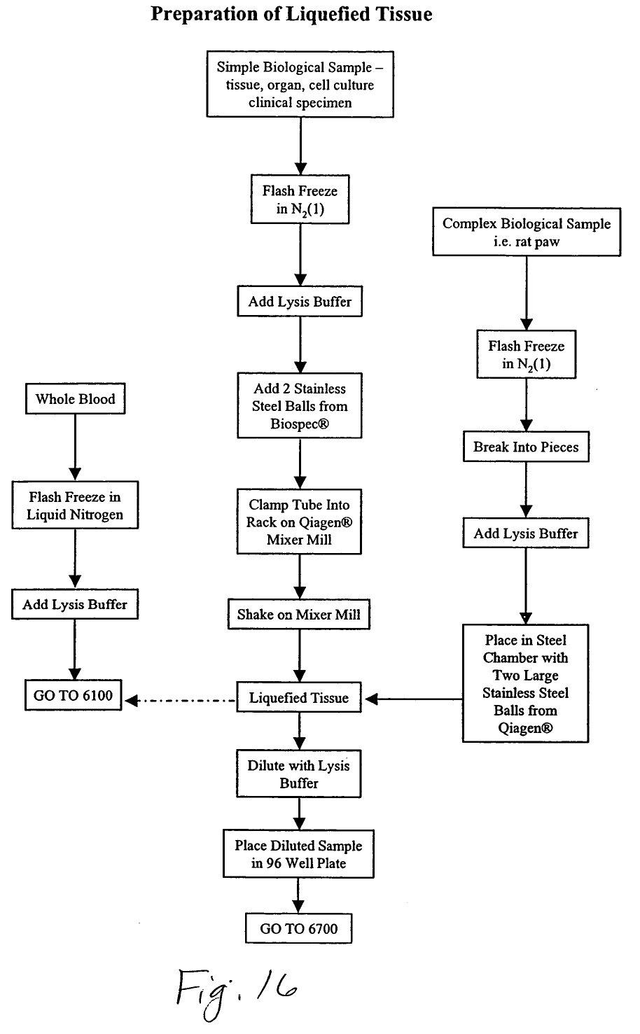

FIG. 16 is a flow diagram for preparation of liquefied tissue for various tissues types. Typically, flash freezing is carried out by placing the sample into ep-tudes prechilled on dry ice and freezing the tube in liquid nitrogen (80 degrees centigrade). While independent protocol is set out for each tissue type, all samples are diluted in a lysis buffer to prevent clogging of downstream filters. While purification is semi-automatic on the ABI 6100, this machine allows multiple loadings during purification and uses the same reagents as the more sophisticated ABI 6700. ABI Prism 6700 is a contained, vaccum-driven unit with HEPA filter that may be used for infectious human samples. The machine will not run unless closed with safety interlock turned over.

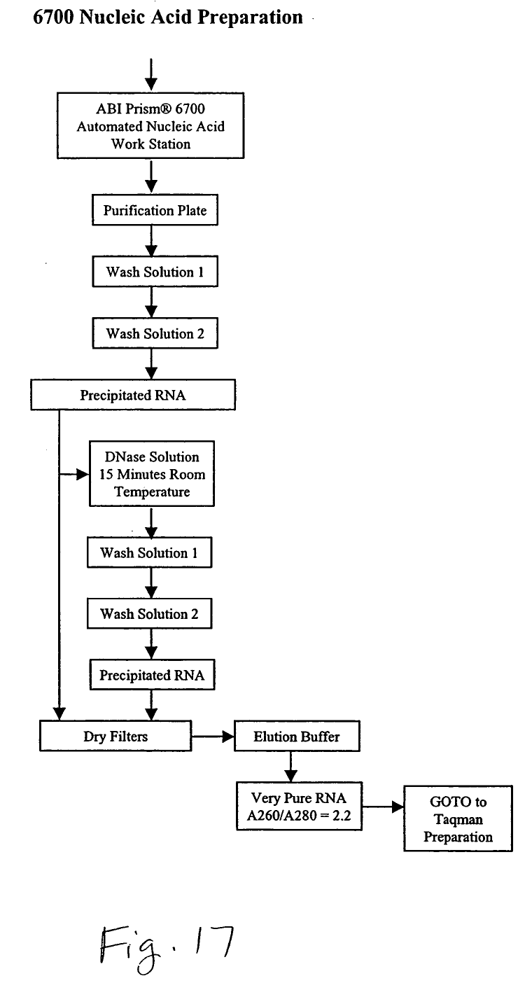

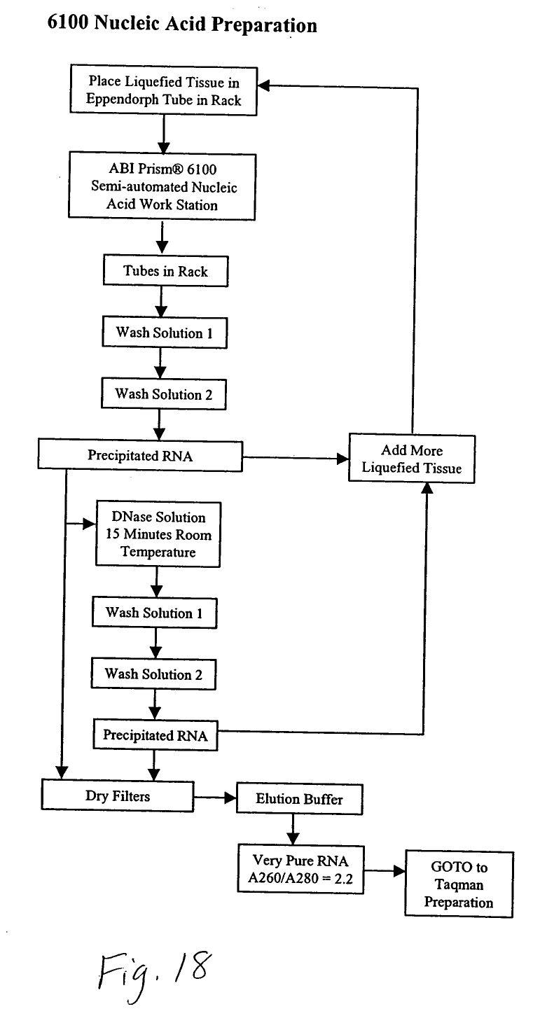

FIG. 17 is a flow diagram of the nuclei acid preparation as performed on an ABI 6700. FIG. 18 is a flow diagram of the nuclei acid preparation as performed on an ABI 6100. Most DNA is removed by wash 1. Wash solution 2 causes an ethanol based precipitation event to occur.

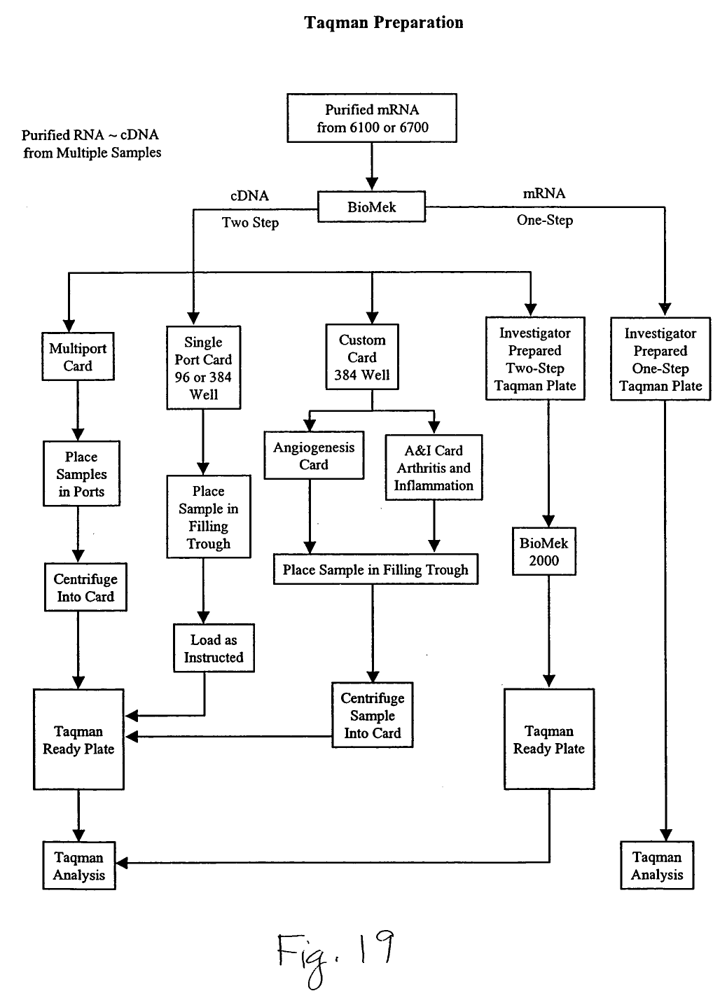

FIG. 19 is a flow diagram of the process of preparing the sample for Taqman analysis. As discussed below, the Biomek is a flexible and easy to use device that supports many users. Biomek includes a multiple pipette head and is useful for 96 well plates or racks of eppendorf tubes. The Biomeck is very fast and can pipette a plate in as little as 10 minutes. The Biomek requires some programming. Although software is provided, the user individualizes the program.

FIG. 20 is a flow diagram of the Taqman analysis prepared in connection with the subject invention. This analysis is particularly suitable for use in connection with the ABI 7900 or ABI 7700 Sequence Detection System and discussed in greater detail above.

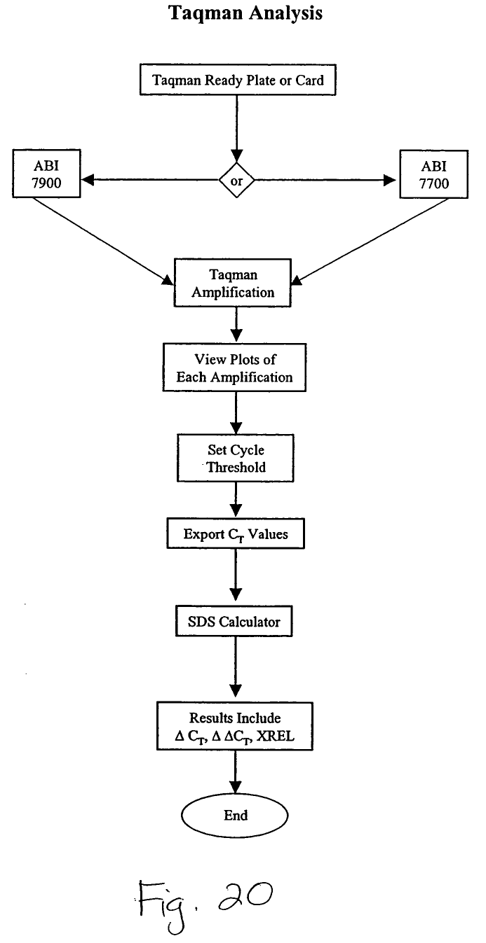

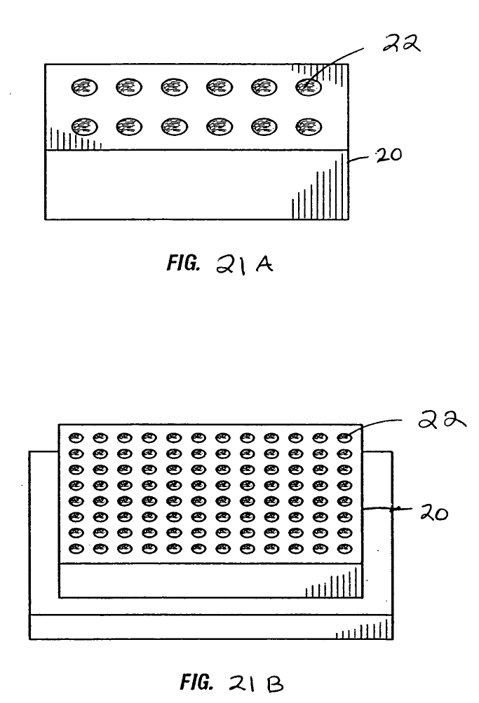

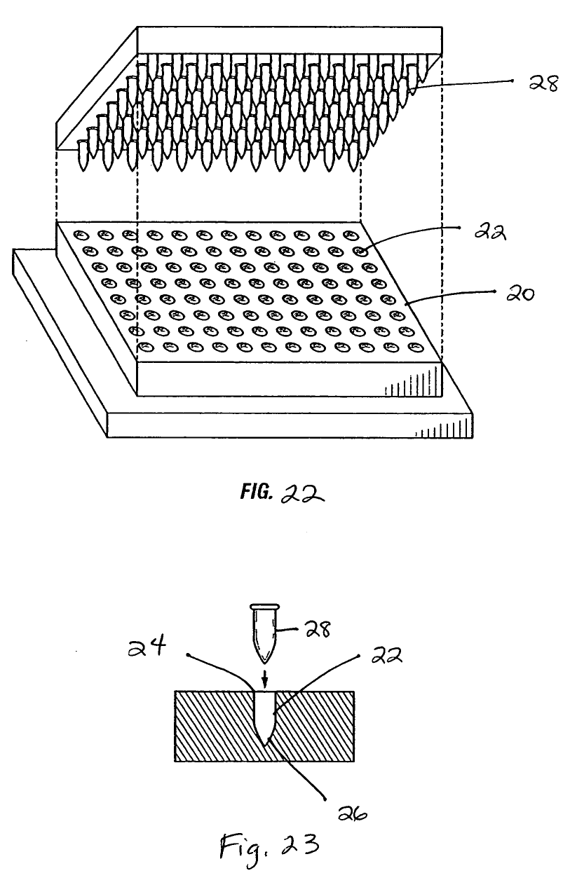

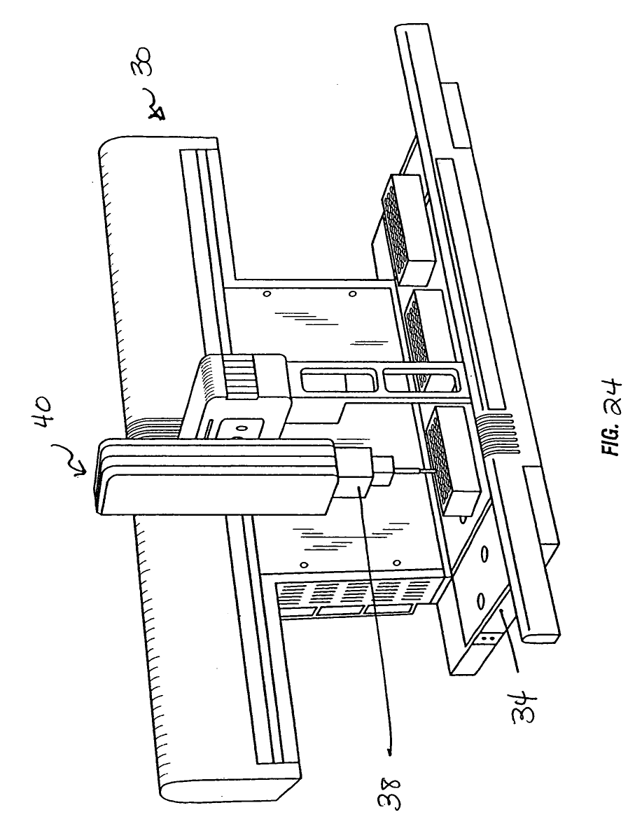

The subject invention also includes an apparatus for maintaining RNA at a temperature between about 0 to 10° C. As disclosed and claimed in U.S. patent application Ser. No. 60/411,174 and as shown in FIGS. 21 through 24, one such device is a metal block 20 for use in a high throughput RNA laboratory comprising a plurality of wells 22. Each well 22 has an open cylindrical upper end 24 and a closed conical lower end 26. Each well 22 is designed to accommodate a biological sample receptacle 28. The receptacle 28 has substantially the same shape as the well, thereby maintaining the temperature of a biological sample in the receptacle during sample set up and prior to polymerase chain reaction. Use of the metal block with an automated liquid handling device 30 and for genetic analysis of biological samples provides an improvement to liquid handling systems currently available.

The metal block 20 is particularly useful for high throughput RNA analysis of a biological sample in combination with an automated liquid handling device. Here, the biological sample is inserted into the biological sample receptacle 28 as held by the wells 22 of the metal block 20 in the automated liquid handling device 30. Subsequently, reverse transcriptase polymerase chain reaction is used to determine the presence of RNA or DNA in the sample via a nucleic acid amplification machine.

An improved automated liquid handling device 30 for genetic analysis of biological samples is also provided. The handling device 30 controls dispensing, aspirating and transferring of liquid from a first microtiter plate well or other biological sample receptacle to a second microtiter plate well or other second biological sample receptacle. The automated liquid handling device is capable of functioning with test tubes, freezing vials, reservoirs and other wet chemistry containers. The improvement to the liquid handling device comprises use of the metal block 20 comprising a plurality of wells 22 where each well 22 has an open cylindrical upper end 24 and a closed conical lower end 26. Each well 22 accommodates a biological sample receptacle 28 having substantially the same shape as the well 22. The biological sample and reagents are pipeted into the receptacle 28 and the temperature of a biological sample during sample set-up and prior to polymerase chain reaction analysis is maintained.

Furthermore, a method of handling a liquid biological sample in a high throughput RNA laboratory is provided. Such method includes the steps of chilling the metal block, inserting the biological sample receptacle into the metal block, positioning the metal block onto an automated liquid handling device and transferring the biological sample into biological sample receptacle in the metal block for polymerase chain reaction analysis.

The metal block of the subject invention is preferably made of aluminum, but may be made of other materials including, but not limited to, copper, gold, or silver. Any material with having high thermal conductivity may be suitable for use in the present invention. The metal block is designed to maintain sample temperature of 0 to 10° C.

The suitable biological sample receptacle includes polypropylene tubes, thermal cycler tubes, a 96 well plate, or a 384-well plate. Biological sample receptacles may be made of plastic or glass. Frequently, biological sample receptacles are plastic and are made of polypropylene or polycarbonate. Thin-walled tubes and plates are preferred as they allow rapid and consistent heat transfer. Tube volume capacity may range from approximately 0.2 milliliters to 1.7 milliliters. Volume capacities of individual microplate tubes vary from approximately 0.2 milliliters in a 96 well format to approximately 0.04 milliliters for the 384 well format.

As discussed above, the biological sample as used herein may be any composition comprising RNA, DNA or genetic sequences created using RNA or DNA from any one or more of the tissues that make up an animal or tissue culture. The tissue from which the RNA originated may include, but are not limited to, epithelial, connective, muscular, and nerve tissues.

To purify a nucleic acid sequence or mRNA, a sample is first collected and liquefied or pulverized. It is important that RNA purification is done by a method that minimizes degradation. The researcher analyzing the results of gene expression must collect and analyze animal tissues as quickly as possible, beginning at the time the animal is euthanized and the organs harvested.

mRNA is subsequently purified using one of a number of methods or devices including a automated nucleic acid workstation such an ABI Prism® 6700. Other devices for purification include but are not limited to the Qiagen BioRobot 9604 or 8000. The technician may also purify the RNA or DNA without using a nucleic acid workstation using alternative purification methods including, but not limited to, glass fiber filter systems such as RNeasy by Qiagen, RNaqueous technology from Ambion, or Absolutely RNA Microprep Kit from Stratagene. RNA may also be purified through precipitation reactions using phenol based products, isopropyl alcohol and lithium chloride. Also, available is a product known as Nucleopin by BD Biosciences.

Following purification of the RNA or DNA, reagents are added to the biological sample in the biological sample receptacle 18 so that the RT-PCR or PCR reaction may occur. Commonly used reverse transcriptases include, but are not limited to, avian myeloblastosis virus (AMV), or Moloney murine leukemia virus (MMLV or MuLV). MMLV and MuLV have lower RNase H activities than AMV but AMV is more stable at higher temperatures. As an alternative, some thermostable DNA polymerases such as Thermus thermophilus DNA polymerase have reverse transcriptase activity in the presence of manganese, allowing for the use of only one enzyme for reverse transcription and polymerase chain reaction. If bicine buffer with manganese is used, intermediate additions between reverse transcription and amplification are not needed and stability at elevated temperatures is not a concern. However the presence of manganese may reduce the fidelity of nucleotide incorporation. Therefore, this method is not suitable for a high throughput RNA analysis. As described in more detail below, other reagents may include, but are not limited to, oligonucleotide primers, a thermostable DNA polymerase and an appropriate reaction buffer such as 500 mM KCl, 100 mM Tris-HCl, 0.1 mM EDTA.

Automated liquid handling devices are often used in laboratories to increase the sample throughput and decrease pipetting error as compared with a human being. These devices are able to transfer reagents from one location to another according to a pre-programmed pattern. The refrigerated table designed to maintain sample temperature table is not satisfactory for maintaining the sample at a sufficient temperature to preserve the activity of the enzyme.

The Beckman Biomek® 2000 is an example of one such device. The Biomek 2000 is an automated liquid handling workstation capable of programmed tasks such as sample pipetting, serial dilution, reagent additions, mixing, reaction timing and similar known manual procedures. The Biomek® 2000 is adapted to aspirate liquid from one location to dispense the liquid in another location automatically in accordance with user programmed instructions. In this liquid handling system, microtiter plates, tip support plates, and troughs are supported in a table attached to the laboratory workstation base. Movement of the table is provided by a motor means causing the table to reciprocally move in at least one axis. A modular pod suspended above the table has an arm attached at one end for movement up and down a vertically extending tower rising from the base of the workstation. The pod is capable of motion along the arm in at least a second axis that is perpendicular to the first axis of movement of the support table. The arm moves up and down in a third direction perpendicular to both the first and second directions.

As more fully described in U.S. Pat. Nos. 5,104,621 and 5,108,703, incorporated herein by reference, the pod is connected with and supports a fluid dispensing, aspirating and transferring means. In the Biomek® 2000, a fluid dispensing pump is connected to the pod by fluid conduits to provide pipetting, dispensing, and aspirating capability. Fluid is dispensed using interchangeable modules of one or more nozzles. The nozzles have pipettor tips affixed to them that are automatically picked up and ejected by the pod.

As shown in FIG. 24, this automated liquid handling device has a table 34, a pod 38 for transferring fluid to a well located on the table 34 and a means 40 for moving the pod relative to the table between selected locations on table 34. The table 34 acts as a surface for supporting the metal block, biological sample receptacles, reagent reservoirs and pipettor tips. The pod 38 is capable of movement horizontally and vertically. The temperature of the table 34 is controllable and is achieved through the use of one or more circulating water baths.

As with many liquid handling devices, the Biomek® 2000 liquid handling device is capable of being programmed to maintain the table at a given temperature and to pipet all reagents required for a given assay into a biological sample receptacle. The device software allows the user to specify the location of the aspiration, dispensation and mixing, what type of labware the liquid is being aspirated from and into and the volume and height of the aspiration and dispensation.

In the subject invention, a biological sample is prepared by liquefying or pulverizing a complex biological construct. RNA is then extracted by one of a variety of methodologies. The metal block 20 having been previously refrigerated or frozen is fixed into position on an automated liquid handling device 30. Biological sample receptacles 18 are then inserted into the metal block 20. As the temperature of the liquefied biological sample is maintained, reagents are added to the liquid biological sample for polymerase chain reaction analysis. Reagents are added into the biological sample receptacles 18 by the automated liquid handling device. The biological sample receptacles are then either moved by robot or manually to a sequence detection system where the reverse transcription, polymerase chain (RT-PCR) reaction amplification and analysis occur.

In another embodiment, the apparatus for maintaining RNA at a temperature between about 0 to 10° C. comprises a combination of devices including an incubator, a quantitative analysis machine and a transfer mechanism for automated transfer of a plate to and from the incubator and to and from the quantitative analysis machine. Here, the plate is maintained in a queue in the incubator prior to analysis in the quantitative machine at a temperature below about 10 degrees centigrade.

Automated liquid handling devices are often used in laboratories to increase the sample throughput and decrease pipetting error as compared with a human being. These devices are able to transfer reagents from one location to another according to a pre-programmed pattern. The Beckman Biomek® 2000 is an example of one such device. The Biomek 2000 is an automated liquid handling workstation capable of programmed tasks such as sample pipetting, serial dilution, reagent additions, mixing, reaction timing and similar known manual procedures. The Biomek 2000 is adapted to aspirate liquid from one location to dispense the liquid in another location automatically in accordance with user programmed instructions.

Other devices that may be used include, but are not limited to, the Qiagen 8000, 3000 or 9600, the Gilson Constellation® 1200 Liquid Handler, the Zymark Sciclone ALH, Staccato® Plate Replication Workstation, or RapidPlate® 96/384 Microplate Pipetting Workstation.

The Qiagen BioRobot 8000 is a nucleic acid purification and liquid handling workstation. It has robotic handling, automated vacuum and a buffer delivery system. Sample receptacles and reagent troughs are present on a platform and an 8 channel pipetting system performs high-speed dispensing. The Qiagen BioRobot 3000 is an automated liquid handling and sample processing workstation. It allows the integration of other hardware, such as cyclers or spectrophotometers. It has fully automated plate processing by transferring labware to various positions on and off of the worktable, as well as temperature control, small volume liquid handling and customizable processing parameters. The Qiagen BioRobot 9600 is an automated workstation for nucleic acid purification, reaction set-up, PCR product clean-up, agarose-gel loading and sample rearray and has a worktable and programmable pipetting mechanism.

The Gilson Constellation 1200 Liquid Handler has a bed that can hold up to 12 microplates, a robotic gripper arm, capability to dispense nanoliter volumes and an optional heating and cooling recirculator.

The Zymark Sciclone ALH Workstation has a 20 position deck; bulk dispensing capabilities to microplates by syringe or peristaltic pump and can pipet using a single channel, 8 channel, 12 channel or 96 channel head. The Robbins Scientific Tango Liquid Handling System comprises a worktable and automated aspiration and dispensing of liquid in a 96 or 384 well format.

All of the devices are able to transfer reagents from one location to another according to a pre-programmed pattern. A refrigerated table to maintain sample temperature may be present upon the device but in a high throughput RNA laboratory, the refrigerated table is not satisfactory for maintaining the sample at a sufficient temperature to preserve the activity of the enzyme, prevent RNA degradation and prevent premature Tag activity.

In the present invention, reagents are added to the biological sample receptacles (also referred to herein as “plates”) positioned on a liquid handling device. The plates may be subsequently positioned on a plate stacker where they are held in a queue. The mechanism of the subject invention transfers the plate from the liquid handling device or plate stacker to an incubator for refrigeration.

Suitable incubators include Cytomat Heraeus sometimes available with internal robots. The incubator of the subject invention is able to maintain the desired temperature of below 10 degrees centigrade. The interior cavity of the incubator is preferably designed with the capability of holding various types of labware. Also, one preferred incubator has a first door for user access to the plates held in queue and a second door where plates may be transferred to and from the incubator. The second door is programmed to open and close when plates are in process of being transferred to and from the incubator. The incubator also preferably comprises an incubator plate handler and incubator dock for loading and unloading plates into and from the incubator. The incubator has the ability to detect when the plate handler of the subject invention approaches the incubator dock, and upon such time, the second door of the incubator is opened for transfer of plates to and from the incubator. The plate handler subsequently transfers the plate from the incubator to a quantitative analysis machine such as sequence detection system where the reverse transcription, polymerase chain (RT-PCR) reaction amplification and analysis occur.

With the appropriate modification, existing plate handlers maybe suitable for use in connection with the subject invention. These plate handlers include the Zymark Twister. One version of the Zymark Twister is taught in U.S. Pat. No. 4,835,711 incorporated herein by reference. The Zymark Twister has a robotic manipulator that individually moves up to 20 plates from each dock. A dock is a vertical column where the plates are stacked. Additional docks may be added.

The plate handler then transfers the plate from the incubator to a plate station on a quantitative analysis machine. Suitable quantitative analysis machines include but are not limited to the ABI Prism 7700 or 7900 sequence detection systems. Other sequence detection systems or devices that perform individual functions of a sequence detection system may be used with the subject invention include but are not limited to a Roche Applied Science LightCycler, BioRad iCycler, MJ Research Opticon, Corbett Rotorgene, Stratagene Mx4000 Multiplex Quantitative PCR System. A fluorimeter and analysis program may be used in connection with devices in which these function are not integrated. The sequence detection system is able to vary reaction conditions to optimize amplification of a nucleic acid sequence, analyze the amount of a given nucleic acid sequence present by detecting fluorescent probes using a fluorescence detection device and analyzing the results via a sequence detection system software.

Once the plate is positioned within the quantitative analysis machine, RT-PCR is then carried out. PCR amplification of a specific DNA segment, referred to as the template, requires that the nucleotide sequence of at least a portion of each end of the template be known. From the template, a pair of corresponding synthetic oligonucleotide primers (“primers”) can be designed. The primers are designed to anneal to the separate complementary strands of template, one on each side of the region to be amplified, oriented with its 3′ end toward the region between the primers. The PCR reaction needs a DNA template along with a large excess of the two oligonucleotide primers, a thermostable DNA polymerase, dNTPs and an appropriate reaction buffer.

PCR amplification of a specific DNA segment, referred to as the template, requires that the nucleotide sequence of at least a portion of each end of the template be known. From the template, a pair of corresponding synthetic oligonucleotide primers (“primers”) can be designed. The primers are designed to anneal to the separate complementary strands of template, one on each side of the region to be amplified, oriented with its 3′ end toward the region between the primers. The PCR reaction needs a DNA template along with a large excess of the two oligonucleotide primers, a thermostable DNA polymerase, dNTPs and an appropriate reaction buffer.

To effect amplification, the mixture is denatured by heat to cause the complementary strands of the DNA template to disassociate. The mixture is then cooled to a lower temperature to allow the oligonucleotide primers to anneal to the appropriate sequences on the separated strands of the template. Following annealing, the temperature of the reaction is adjusted to an efficient temperature for 5′ to 3′ DNA polymerase extension of each primer into the sequences present between the two primers. This results in the formation of a new pair of complementary strands. The steps of denaturation, primer annealing and polymerase extension can be repeated many times to obtain a high concentration of the amplified target sequence. Each series of denaturation, annealing and extension constitutes one “cycle.” There may be numerous “cycles.” The length of the amplified segment is determined by the relative positions of the primers with respect to each other, and therefore, this length is a controllable parameter. By virtue of the repeating aspect of the process, the method is referred to as the “polymerase chain reaction” (hereinafter “PCR”).

As the desired amplified target sequence becomes the predominant sequence in terms of concentration in the mixture, this sequence is said to be PCR amplified. With PCR, it is possible to amplify a single copy of a specific target DNA sequence to a level detectable by several different methodologies. These methodologies include ethidium bromide staining, hybridization with a labeled probe, incorporation of biotinylated primers followed by avidin-enzyme conjugate detection, and incorporation of ³²P-labeled deoxynucleotide triphosphates such as Dctp or Datp into the amplified segment.

The development of real-time PCR, also known as kinetic PCR, has provided an improved method for the quantification of specific nucleic acids. In real-time PCR, cycle-by-cycle measurement of accumulated PCR product is made possible by combining thermal cycling and fluorescence detection of the amplified product in a single instrument. Because the product is measured at each cycle, product accumulation can be plotted as a function of cycle number. The exponential phase of product amplification is readily determined and used to calculate the amount of template present in the original sample. A number of alternative methods are currently available for real-time PCR.

The original protocol developed by Grossman et al. (U.S. Pat. No. 5,470,705, hereby incorporated by reference) used radioactive labels on the probes but further refinements of the method have focused on self-quenching fluorescent probes. Originally, separation of the amplified products by electrophoresis or other methods was used to measure and calculate the amount of released label. This added time-consuming steps to the analysis. Furthermore, this end-stage analysis of the reactions cannot be readily applied to real-time PCR.

In one current method, fluorogenic exonuclease probes for the real-time detection of PCR products are used. This type of technology is captured in the ABI Prism® 7700 Sequence Detection System and disclosed in Livak et al (U.S. Pat. No. 5,538,848 hereby incorporated by reference). In a modification of an existing method utilizing radioactive labels, fluorogenic exonuclease probes are designed to anneal to sequences between the two amplification primers but contain one or more nucleotides that do not match at the 5′ end. The nonmatching nucleotides are linked to a fluorescence donor. A fluorescence quencher is positioned typically at the end of the probe. When the donor and quencher are in the same vicinity, the quencher prevents the fluorescence donor from emitting light.

Traditional fluorescence quenchers absorb light energy emitted by an excited reporter molecule and release this energy by fluorescing at a higher wavelength. Increased sensitivity in real-time detection can be achieved with dark quenchers such as dabcyl or the developed Eclipse Quencher from Epoch Biosciences, Inc. The dark quenchers absorb fluorescent energy but do not fluoresce themselves, thus reducing background fluorescence in the sample. The dark quencher works effectively against a number of red-shifted fluoropores such as FAM, Cy3 and Tamra due to its broader range of absorbance over dabcyl (400-650 nm versus 360-500 nm respectively) and is thus better suited to multiplex assays.

The sensitivity of real-time PCR can also be augmented through the use of minor groove binders (“MGBs”) (also from Epoch Biosciences, Inc.), which are certain naturally occurring antibiotics and synthetic compounds able to fit into the minor groove of double-stranded DNA to stabilize DNA duplexes. The minor groove binders can be attached to the 5′ end, 3′ end or an internal nucleotide of oligonucleotides to increase the oligonucleotide's temperature of melting, i.e., the temperature at which the oligonucleotide disassociates from its target sequence and hence creates stability. The use of MGBs allows for the use of shorter oligonucleotide probes as well as the placement of probes in AT-rich sequences without any loss in oligonucleotidal specificity, as well as better mismatch discrimination among closely related sequences. Minor groove binders may be used in connection with dark quenchers or alone.

Thermus aquaticus (taq) DNA polymerse used for the PCR amplification has the ability to cleave unpaired nucleotides off of the 5′ end of DNA fragments. In the PCR reaction, the fluorogenic probe anneals to the template (the nucleotide sequence of interest in a sample). An extension of both primers and the probe occurs until one of the amplification primers is extended to the probe. Taq polymerase then cleaves the nonpaired nucleotides from the 5′ end of the probe, thereby releasing the fluorescence donor. Once it is physically separated from the quencher, the fluorescent donor can fluorescence in response to light stimulation. Because of the role of taq polymerase in this process, these probes are often referred to as TaqMan® probes. As more PCR product is formed, more fluorescent donors are released, allowing the formation of the PCR product to be measured and plotted as a function of cycle time. The linear, exponential phase of the plot can be selected and used to calculate the amount of nucleotide in the sample. The development of these self-quenching fluorescent probes was a considerable advancement in quantitative PCR. Numerous improved self-quenching probes and methods for the use thereof have been subsequently reported in U.S. Pat. Nos. 5,912,148, 6,054,266 (Kronick et al.) and 6,130,073 (Eggerding).

The LightCycler® uses hybridization instead of exonuclease cleavage to quantify the amplification reaction. This method also adds additional fluorogenic probes to the PCR amplification. However, unlike the TaqMan® system, fluorescence increases in this system when two different fluorogenic probes are brought together on the same template by extension or hybridization, allowing resonance energy transfer to occur between the two probes.

Other systems are also available. The Amplifluor® primers produced by Intergen® are hairpin oligonucleotides, which form hairpins when they are single-stranded, which bring a fluorescence donor and quencher into close proximity. When the primers are incorporated into a double stranded molecule, the hairpins are straightened, which separates the donor and quencher to cause an increase in fluorescence. Other applications use intercalating dyes, which only associate with double stranded DNA. As more double stranded DNA is generated by the reaction, more fluorescence is observed as more dye becomes associated with DNA. Regardless of the method used, the end result is the same, a plot of fluorescence versus cycle number. Further analysis of this data is then used to derive quantitative values for the RNA present in the samples. Hence, amplified segments created by the PCR process are efficient templates for subsequent PCR amplifications leading to a cascade of further amplification.

The amplification of nucleic acid sequences may occur within and be analyzed by a sequence detection system, such as the ABI Prism® 7900. The sequence detection system is able to vary reaction conditions to optimize amplification of a nucleic acid sequence. The system can analyze the amount of a given nucleic acid sequence present using any number of fluorescent probes, a fluorescence detection mechanism and system software. Other devices that may be used to provide temperature cycling with or without detection capabilities including but are not limited to a Roche Applied Science LightCycler®, BioRad Cycler, MJ Research Opticon, Corbett Rotorgene, and Stratagene Mx4000® Multiplex Quantitative PCR System. A fluorimeter and analysis program may be used in conjunction with devices in which these functions are not integrated. The sequence detection system is able to vary reaction conditions to optimize amplification of a nucleic acid sequence. The system can analyze the amount of a given nucleic acid sequence present using any number of fluorescent probes, a fluorescence detection mechanism and sequence detection system software.

Detailed embodiments of the present invention are disclosed herein. However, it is to be understood that the disclosed embodiments are merely exemplary of the invention that may be embodied in various and alternative forms. The figures are not necessarily to scale where some features may be exaggerated or minimized to show details of particular components. Therefore, specific structural and functional details disclosed herein are not to be interpreted as limiting, but merely as a basis for the claims and as a representative basis for teaching one skilled in the art to variously employ the present invention.

Although making and using various embodiments of the present invention have been described in detail above, it should be appreciated that the present invention provides many applicable inventive concepts that can be embodied in a wide variety of specific contexts. The specific embodiments discussed herein are merely illustrative of specific ways to make and use the invention, and do not delimit the scope of the invention.

We claim:

1. A method of analyzing RNA comprising the steps of:
extracting RNA from the complex biological construct in sufficient quantities to detect RNA,
transferring the RNA to an apparatus for maintaining the RNA at a temperature between about 0 to 10° C.; and
analyzing RNA levels and function with a computer generated mathematical analysis.

2. The method of claim 1 further comprising the step of isolating and purifying the RNA.

3. A method of analyzing RNA comprising the steps of:
pulverizing a complex biological construct,
extracting RNA from the complex biological construct in sufficient quantities to detect RNA,
transferring the RNA to an apparatus for maintaining the RNA at a temperature between about 0 to 10° C.; and
analyzing RNA levels and function with a computer generated mathematical analysis.

4. The method of claim 3 further comprising step of isolating and purifying the RNA.

5. A high throughput RNA laboratory comprising
an apparatus for extracting nucleic acids from a complex biological construct;
an automated nucleic acid workstation for isolating and purifying RNA from said complex biological construct;
an apparatus for maintaining said RNA samples at a temperature of between about 0 to 10° C., and
a computer readable program for use in connection with an information display apparatus wherein said computer readable program causes a computer to calculate and display RNA data.

6. The high throughput RNA laboratory wherein the RNA data includes cycle threshold valves, a delta C_{T}, a delta delta C_{T} and a relative transcription change (XRel) of said RNA sample provided by a real-time quantitative PCR amplification system.

7. The high throughput RNA laboratory of claim 5 further comprising an automatic liquid-handling apparatus for preparing RNA samples for reverse transcription and PCR amplification.

8. The high throughput RNA laboratory of claim 5 further comprising a real-time quantitative PCR amplification system.
